SEQUENCE LISTING (26 sequences)

SEQ ID NO: 1 - DNA, 1011 bp, Helianthus annuus
gtcgagctcgtctcgtaaaatgttcgagtcagctccaattaaatcatgtcggcttgttatattttttttaatttatttttgatattttttacatatatttataacataaaaaacaaaataaaaataaaaattacacatatatctatatgtattatttttctaaaattttaaatagcgaaagacatattaaaagtattatatgtataattttgtttagcttcccatatttttatatgttattaattaattaaacttaaaatgttaacactttaacacctcttacatactttttagttcaatacatttaaaattaaaattaatctatatgcaataaaataattcaagcaggcttgcaagctcacgagtcgagccatgcctggctcgagctcgactcatttacaaatcgagccaccaagccgactcgtttataaccgagttttttttagccgagtttttttcaagcgaacttcaaacaagtcacgcgcttttaattaacacattctagtctaaagaagataattgaaagagaaagtagatataagtaaaaggagtagccaaagatataaatttagggtctaaacaacctaatattgtttaattttttttaaataaactagtttttttttaccgattatctgtgttatatgtcttagtttgacatgataagttatcataattacttgtagtatttttatatcagaaatatacgttggagaattaaattttatcctgatcgtcaattgacaagaacaaaaatcaacatctcatggttttttactaatttatatgattaaagatatatggttgtaagaaaaagaacaatgtacatcaaatggtgaaatttgaatatttgatagtaacgtaatccattgtgtatttcttattattttatcattttcccaaggtgtgtcatatatagtgtctccattctttctatagcacaatatccttcacctccctctctctctctctctctctaaaaatgatgatgagacgacaaagatcgaattc

SEQ ID NO: 2 - RNA, 1195 nt, Helianthus annuus
uugaaauucugagaaaagccacauaaucaaagcuaaagaggugguuuaaacagcugaugacuugcacuggaauggcuuucuucuccuccaauuucauguuacaauccucccaagaagaugaccaucaugccccuacaucucucucuccaauccucccaccuugcaguaccaccacucaagauuucaguggugcugcuuucuugggaaaaagaucuaugucuucuuacucagguuugaacaacaacaacauggauggaugugaucaagaagggaacaugaauggagaagaugaguuaucagaugauggaucacagcuucuugcaggagagaaaaagaggagauuaaacauggaacaagugaagacacuugagagaaacuuugaguuaggaaauaagcuugaaccugagaggaaaaugcaacuugcaagagcacuuggacuacaaccaagacagauugcuauaugguuucaaaacagaagagcuagauggaaaacuaaacaguuggaaaaagacuaugaugcccucaagagacaguuugaagcuguuaaagcugagaaugauucacuccaaucucaaaaucauaaacuucaugcugagauaauggcacuaaaaaauagggagccagcagaacuaaucaaccucaacauaaaagaaacagaaggaucuugcagcaaccgaagcgaaaacagcucugaaaucaaacuagacaucucaagaacaccggcuaccgauagcccuuuaucaucacaccaucaacaccaacaccagccaauaccuaaucuuuuuccaucgucgaauaucgauaggccuaauucgaauaacauuguggcgcaucaacuuuuccacaauucgucaucaaggccggcagaucaucaacuucauugccacaaacucgaucaaucgaaugccauuaaagaagaauguuuuagcacaauguuuguugguauggaugaucaaucaggguuuuggccaugguuggaacaaccacaauucaauugauggaaucaagaagcaaaaaagcaaaagaaaacgguacccgauucgccuucuuggcuuugguuugauuauauuaaagauggagaucaucaaucuguuuguucucuaagcuuuaaauucuuguuuuuugguacuuaaauuaauagaguaaaaauuagaagaaaaaacguauuauuauuuuaaauucaagauuaguguuu

SEQ ID NO: 3 - DNA, 315 bp, Helianthus annuus
aattaactcaccttaactaagttacttatgacaacatctctctcatagatcttgatgcagcttgcattcatgagttgtgatgtacaactcattcatgcattagggtttcagttttttcaaagttttttttttattttttcttctgtttcaagatcatgatgatgagttgtgctgaacacttgaacagctcattgatgcattagggtttgttttagtttcaagttctttcttttctttcattttcatgcactaaatccatatgggcttgaagaaagtttgaatctttatatgttagttgatgatcttgatgcaggt

SEQ ID NO: 4 - DNA, 778 bp, Helianthus annuus
gtaatattagtttgattgtttattgcatctatcaatcattagattctactctttacttgatcacacagaaagtaactaaaccttttttcctaatgataacaatatttgttttgcaaatctaatggcaatcaaataaaagtttctggtaagcagccatgatctatttatttttcactatttgagtaagtttaaaagttgcatttatcctcactaattatatacaacactaaaataatcattaaactgactgttataattactttccgtaaacggtatgccaaaacttaaaatgattaacaattttataagaatggaaagtaaaatcattacactatttcccatattagtcatgaccaaagtttgtttctttctgaagggcaaaagggtcaatatgcttatatgcagcatgggcaaaagaagtagagtgtatatcaaaattcatatctttattttcttttcaaagtttaggtaacaaaaagaagaaattataaacgagtttgttacaattccacaagtacatgaagaaacaaaatttgttagtatttttattttccatgtttttagtaacttccatatcaatttagcactagaagataactttttttaggactcggtaaaccatacaagtagggtcatactttatcgtttatccattaatgtatatccataaattcactgattatgcggtatttcctttgttacactgtcttgaacaagtattagtacatgtagtttcttaaagattgtttaatcaaccaaaaagattgaaactttgcag

SEQ ID NO: 5 - protein, 313 aa, Helianthus annuus
MTCTGMAFFSSNFMLQSSQEDDHHAPTSLSPILPPCSTTTQDFSGAAFLGKRSMSSYSGLNNNNMDGCDQEGNMNGEDELSDDGSQLLAGEKKRRLNMEQVKTLERNFELGNKLEPERKMQLARALGLQPRQIAIWFQNRRARWKTKQLEKDYDALKRQFEAVKAENDSLQSQNHKLHAEIMALKNREPAELINLNIKETEGSCSNRSENSSEIKLDISRTPATDSPLSSHHQHQHQPIPNLFPSSNIDRPNSNNIVAHQLFHNSSSRPADHQLHCHKLDQSNAIKEECFSTMFVGMDDQSGFWPWLEQPQFN

SEQ ID NO: 6 - DNA, 1195 bp, Helianthus annuus
ttgaaattctgagaaaagccacataatcaaagctaaagaggtggtttaaacagctgatgacttgcactggaatggctttcttctcctccaatttcatgttacaatcctcccaagaagatgaccatcatgcccctacatctctctctccaatcctcccaccttgcagtaccaccactcaagatttcagtggtgctgctttcttgggaaaaagatctatgtcttcttactcaggtttgaacaacaacaacatggatggatgtgatcaagaagggaacatgaatggagaagatgagttatcagatgatggatcacagcttcttgcaggagagaaaaagaggagattaaacatggaacaagtgaagacacttgagagaaactttgagttaggaaataagcttgaacctgagaggaaaatgcaacttgcaagagcacttggactacaaccaagacagattgctatatggtttcaaaacagaagagctagatggaaaactaaacagttggaaaaagactatgatgccctcaagagacagtttgaagctgttaaagctgagaatgattcactccaatctcaaaatcataaacttcatgctgagataatggcactaaaaaatagggagccagcagaactaatcaacctcaacataaaagaaacagaaggatcttgcagcaaccgaagcgaaaacagctctgaaatcaaactagacatctcaagaacaccggctaccgatagccctttatcatcacaccatcaacaccaacaccagccaatacctaatctttttccatcgtcgaatatcgataggcctaattcgaataacattgtggcgcatcaacttttccacaattcgtcatcaaggccggcagatcatcaacttcattgccacaaactcgatcaatcgaatgccattaaagaagaatgttttagcacaatgtttgttggtatggatgatcaatcagggttttggccatggttggaacaaccacaattcaattgatggaatcaagaagcaaaaaagcaaaagaaaacggtacccgattcgccttcttggctttggtttgattatattaaagatggagatcatcaatctgtttgttctctaagctttaaattcttgttttttggtacttaaattaatagagtaaaaattagaagaaaaaacgtattattattttaaattcaagattagtgttt

SEQ ID NO: 7 - DNA, 3299 bp, Helianthus annuus
gtcgagctcgtctcgtaaaatgttcgagtcagctccaattaaatcatgtcggcttgttatattttttttaatttatttttgatattttttacatatatttataacataaaaaacaaaataaaaataaaaattacacatatatctatatgtattatttttctaaaattttaaatagcgaaagacatattaaaagtattatatgtataattttgtttagcttcccatatttttatatgttattaattaattaaacttaaaatgttaacactttaacacctcttacatactttttagttcaatacatttaaaattaaaattaatctatatgcaataaaataattcaagcaggcttgcaagctcacgagtcgagccatgcctggctcgagctcgactcatttacaaatcgagccaccaagccgactcgtttataaccgagttttttttagccgagtttttttcaagcgaacttcaaacaagtcacgcgcttttaattaacacattctagtctaaagaagataattgaaagagaaagtagatataagtaaaaggagtagccaaagatataaatttagggtctaaacaacctaatattgtttaattttttttaaataaactagtttttttttaccgattatctgtgttatatgtcttagtttgacatgataagttatcataattacttgtagtatttttatatcagaaatatacgttggagaattaaattttatcctgatcgtcaattgacaagaacaaaaatcaacatctcatggttttttactaatttatatgattaaagatatatggttgtaagaaaaagaacaatgtacatcaaatggtgaaatttgaatatttgatagtaacgtaatccattgtgtatttcttattattttatcattttcccaaggtgtgtcatatatagtgtctccattctttctatagcacaatatccttcacctccctctctctctctctctctctaaaaatgatgatgagacgacaaagatcgaattcttgaaattctgagaaaagccacataatcaaagctaaagaggtggtttaaacagctgatgacttgcactggaatggctttcttctcctccaatttcatgttacaatcctcccaagaagatgaccatcatgcccctacatctctctctccaatcctcccaccttgcagtaccaccactcaagatttcagtggtaattaactcaccttaactaagttacttatgacaacatctctctcatagatcttgatgcagcttgcattcatgagttgtgatgtacaactcattcatgcattagggtttcagttttttcaaagttttttttttattttttcttctgtttcaagatcatgatgatgagttgtgctgaacacttgaacagctcattgatgcattagggtttgttttagtttcaagttctttcttttctttcattttcatgcactaaatccatatgggcttgaagaaagtttgaatctttatatgttagttgatgatcttgatgcaggtgctgctttcttgggaaaaagatctatgtcttcttactcaggtttgaacaacaacaacatggatggatgtgatcaagaagggaacatgaatggagaagatgagttatcagatgatggatcacagcttcttgcaggagagaaaaagaggagattaaacatggaacaagtgaagacacttgagagaaactttgagttaggaaataagcttgaacctgagaggaaaatgcaacttgcaagagcacttggactacaaccaagacagattgctatatggtttcaaaacagaagagctagatggaaaactaaacagttggaaaaagactatgatgccctcaagagacagtttgaagctgttaaagctgagaatgattcactccaatctcaaaatcataaacttcatgctggtaatattagtttgattgtttattgcatctatcaatcattagattctactctttacttgatcacacagaaagtaactaaaccttttttcctaatgataacaatatttgttttgcaaatctaatggcaatcaaataaaagtttctggtaagcagccatgatctatttatttttcactatttgagtaagtttaaaagttgcatttatcctcactaattatatacaacactaaaataatcattaaactgactgttataattactttccgtaaacggtatgccaaaacttaaaatgattaacaattttataagaatggaaagtaaaatcattacactatttcccatattagtcatgaccaaagtttgtttctttctgaagggcaaaagggtcaatatgcttatatgcagcatgggcaaaagaagtagagtgtatatcaaaattcatatctttattttcttttcaaagtttaggtaacaaaaagaagaaattataaacgagtttgttacaattccacaagtacatgaagaaacaaaatttgttagtatttttattttccatgtttttagtaacttccatatcaatttagcactagaagataactttttttaggactcggtaaaccatacaagtagggtcatactttatcgtttatccattaatgtatatccataaattcactgattatgcggtatttcctttgttacactgtcttgaacaagtattagtacatgtagtttcttaaagattgtttaatcaaccaaaaagattgaaactttgcagagataatggcactaaaaaatagggagccagcagaactaatcaacctcaacataaaagaaacagaaggatcttgcagcaaccgaagcgaaaacagctctgaaatcaaactagacatctcaagaacaccggctaccgatagccctttatcatcacaccatcaacaccaacaccagccaatacctaatctttttccatcgtcgaatatcgataggcctaattcgaataacattgtggcgcatcaacttttccacaattcgtcatcaaggccggcagatcatcaacttcattgccacaaactcgatcaatcgaatgccattaaagaagaatgttttagcacaatgtttgttggtatggatgatcaatcagggttttggccatggttggaacaaccacaattcaattgatggaatcaagaagcaaaaaagcaaaagaaaacggtacccgattcgccttcttggctttggtttgattatattaaagatggagatcatcaatctgtttgttctctaagctttaaattcttgttttttggtacttaaattaatagagtaaaaattagaagaaaaaacgtattattattttaaattcaagattagtgttt

SEQ ID NO: 8 - protein, 122 aa, Helianthus annuus
LINLNIKETEGSCSNRSENSSEIKLDISRTPATDSPLSSHHQHQHQPIPNLFPSSNIDRPNSNNIVAHQLFHNSSSRPADHQLHCHKLDQSNAIKEECFSTMFVGMDDQSGFWPWLEQPQFN

SEQ ID NO: 9 - protein, 35 aa, Helianthus annuus
LINLNIKETEGSCSNRSENSSEIKLDISRTPATDS

SEQ ID NO: 10 - protein, 29 aa, Helianthus annuus
IKEECFSTMFVGMDDQSGFWPWLEQPQFN

SEQ ID NO: 11 - protein, 34 aa, Helianthus annuus
SINLNKETEGSCSNRSENSSDIKLDISRTPAIDS

SEQ ID NO: 12 - protein, 29 aa, Helianthus annuus
VKEESLSNMFCGIDDQSGFWPWLEQQHFN

SEQ ID NO: 13 - protein, 91 aa, Helianthus annuus
MTCTGMAFFSSNFMLQSSQEDDHHAPTSLSPILPPCSTTTQDFSGAAFLGKRSMSSYSGLNNNNMDGCDQEGNMNGEDELSDDGSQLLAGE

SEQ ID NO: 14 - protein, 57 aa, Helianthus annuus
KKRRLTDEQVKALEKSFELENKLEPERKVQLARELGLQPRQVAVWFQNRRARWKTKQ

SEQ ID NO: 15 - DNA, 1813 bp, Arabidopsis thaliana
accagaagtggtatagtctaggccgatacatttcactatctctctctcttttgtttttcctcttcttctttttttccatttgatttcaaactctcacacaaagagcttcagatttataagaccatgataatggctttaagacaaagattggcaagaagaaaaaactaaagagaaacgaccaaaatctcaagcaaacagtactaacttctgttgcaaaacagaagaagatgtcttgtaataatggaatgtcttttttcccttcaaatttcatgatccaaacctcttacgaagatgatcatcctcatcaatctccatctcttgctcctcttcttccttcttgctctctacctcaagatctccatggtatatatacataaacttccacacacatctcctctgttttctctctatctctttctaatgctctgttctgttctgtttcaggatttgcttcgtttctaggtaagagatctccaatggaagggtgttgtgatttagaaacagggaacaatatgaatggagaagaggattattcagatgatgggtcacaaatgggagagaagaagaggagattgaacatggaacaagtgaagacactagagaagaactttgagcttggaaacaaacttgaaccagagaggaaaatgcagctagctcgtgccttaggtttgcaaccaagacagatcgcgatttggtttcaaaatcgaagagctcgttggaaaacaaagcagctagagaaagattatgatactcttaaacgacagtttgatacacttaaagctgaaaatgatcttcttcaaactcataatcagaaactccaagctgaggtaattaatctcataaattaacaaaaaaaatcaatatgtgttatttttttttgggttaatgatcaataattacagttattttccatctaaaggatgatttttttctttttaaaaaaggttaaaaattatatttctggtttataattatttggatcaggagttgctttcaggtagggttaaaaaactggacatgattcatgacttttcagacatcattatctctttttttcttcactcttgtctggaaagagatctgaaaacaatagtttctttatgcttatcacattgtacagtaactctgtttatgtttaaaattttgtctttaattacgcagataatgggattaaaaaacagagaacaaacagaatcaataaatctaaacaaagaaactgaaggatcttgcagtaacagaagtgataacagttcagataatctcagactagatatctcaactgcgccgccatcaaacgacagtacattaaccggtggccacccaccgccaccacagacagttggtcgacacttcttcccaccgtcgccagccaccgcaacgacaactactacaacaatgcagttctttcaaaactcatcttcaggacagagtatggttaaagaagagaatagtatcagtaacatgttctgtgcaatggatgaccattctggtttttggccatggcttgatcagcaacagtacaattgaaattggtctacctgttttttttgtttttgtttttaaaaaaatttatattttttttttttgtatttggaattttgatcagaagaacccatgcatgttttcaaaaactggaatctatatcattagctcactttgaaatctgcaaccaaacaccactgaggttttttgtttactttttgagtaaatgagatgtaaaaaaatgggtaatatccattatattatataaaaaataatatcattatggcccaacatttttctgtatggagaaaaataaaataaattgtatatt

SEQ ID NO: 16 - protein, 294 aa, Arabidopsis thaliana
MSCNNGMSFFPSNFMIQTSYEDDHPHQSPSLAPLLPSCSLPQDLHGFASFLGKRSPMEGCCDLETGNNMNGEEDYSDDGSQMGEKKRRLNMEQVKTLEKNFELGNKLEPERKMQLARALGLQPRQIAIWFQNRRARWKTKQLEKDYDTLKRQFDTLKAENDLLQTHNQKLQAEIMGLKNREQTESINLNKETEGSCSNRSDNSSDNLRLDISTAPPSNDSTLTGGHPPPPQTVGRHFFPPSPATATTTTTTMQFFQNSSSGQSMVKEENSISNMFCAMDDHSGFWPWLDQQQYN

SEQ ID NO: 17 - DNA, 21 bp, Artificial
  Primer H1qF
ggccggcagatcatcaacttc

SEQ ID NO: 18 - DNA, 21 bp, Artificial
  Primer H1qR
ccaaccatggccaaaaccctg

SEQ ID NO: 19 - DNA, 18 bp, Artificial
  Primer A13qF
ctccatggatttgcttcg

SEQ ID NO: 20 - DNA, 18 bp, Artificial
  Primer A13qR
tcccatttgtgacccatc

SEQ ID NO: 21 - DNA, 24 bp, Artificial
  Primer qAtPR2 - F
taagaaggaaccaacgtatgagaa

SEQ ID NO: 22 - DNA, 24 bp, Artificial
  Primer qAtPR2 - R
cataaaaagcccacaagtctctaa

SEQ ID NO: 23 - DNA, 25 bp, Artificial
  Primer qAtPR4 - F
gtttaagggtgaagaacacaagaac

SEQ ID NO: 24 - DNA, 24 bp, Artificial
  Primer qAtPR4 - R
attgaacattgctacatccaaatc

SEQ ID NO: 25 - DNA, 24 bp, Artificial
  Primer qAtGLU - F
accaaaacctctttgacgctttac

SEQ ID NO: 26 - DNA, 24 bp, Artificial
  Primer qAtGLU - R
aatacgtttccactcctcttccag

(19) United States
(12) Patent Application Publication
(10) Pub. No.: US 2015/0247160 A1
(43) Pub. Date: Sep. 3, 2015

(54) STRESS TOLERANCE IN PLANTS

(71) Applicants: Universidad Nacional Del Litoral, Santa Fe (AR); Consejo Nacional De Investigaciones Cientificas Y Tecnicas, Cdad. de Buenos Aires (AR)

(72) Inventors: Julieta Virginia Cabello, Sante fe (AR); Raquel Lia Chan, Santa Fe (AR)

(21) Appl. No.: 14/384,561

(22) PCT Filed: Mar. 12, 2012
(86) PCT No.: PCT/GB2013/050600

§ 371 Date: Dec. 31, 2014

(30) Foreign Application Priority Data
Mar. 12, 2012 (GB) 1204304.8

(51) Int. Cl. C12N 15/82

(52) U.S. Cl. CPC C12N 15/8273; C12N 15/8271; C12N 15/8279; C12N 15/8237

(57) ABSTRACT

We provide methods for increasing yield in plants under moderate stress conditions by expression of a transcription factor gene belonging to the HD Zip family of transcription factors.

### CROSS-REFERENCE TO RELATED APPLICATIONS

This application is a U.S. national phase application, filed in accordance with 35 U.S.C. §371, of International Application No. PCT/GB2013/050600, filed Mar. 12, 2013, which claims the benefit of the priority of Great Britain Application No. 1204304.8, filed Mar. 12, 2012.

### SEQUENCE LISTING

The instant application contains a Sequence Listing which has been submitted in ASCII format via EFS-Web and is hereby incorporated by reference in its entirety. The ASCII copy, created on May 18, 2015, is named “SequenceListing5D107002US1.txt” and is 23.6 KB (24,249 bytes) in size.

### BACKGROUND OF THE INVENTION

Adverse climate conditions and human activity as well as biological agents are stress effectors for plants and seriously affect their productivity and survival. Losses in productivity due to this kind of stress reach sometimes more than 50%. Plant breeders have been and are devoted to developing strategies in order to avoid or diminish the negative impact of these situations.

Among abiotic stress-causing factors, drought, salinity of the soil and extreme temperatures are some of the most harmful. Temperatures stresses are classified into three types: freezing (caused by temperatures below 0° C.), chilling (caused by low temperatures over 0° C.) and heat stress (caused by high temperatures). Chilling temperatures cause damage to photosynthetic tissues, inhibit the whole photosynthetic process and carbohydrate transport as well as protein biosynthesis and respiration rates. Simultaneously, protein degradation is accelerated. All of these effects occur rather slowly and involve partial or total loss of membrane functionality. In contrast, freezing temperatures cause quick damage, killing the plants. It has been observed, however, that plants subjected to chilling during several days tolerate freezing temperatures better than plants subjected to freezing without first having been exposed to a period of chilling; this process is termed “acclimation”.

Species such as winter cereals are adapted to cold or moderate-cold weather and can tolerate temperatures ranging from 0° C. to 15° C., as well as freezing temperatures, rather well if they have previously been acclimated to reduced temperatures. By contrast, tropical and subtropical species, including important crops, such as maize, rice or tomato, are sensitive to low temperatures and appear to lack efficient acclimation mechanisms.

Furthermore, in Arabidopsis research, stress is often assessed under severe conditions that are lethal to wild type plants. For example, drought tolerance is assessed predominantly under quite severe conditions in which plant survival is scored after a prolonged period of soil drying. However, in temperate climates, limited water availability rarely causes plant death, but restricts biomass and seed yield. Moderate water stress, that is suboptimal availability of water for growth can occur during intermittent intervals of days or weeks between irrigation events and may limit leaf growth, light interception, photosynthesis and hence yield potential. Leaf growth inhibition by water stress is particularly undesirable during early establishment.

In a recent study, different transgenic Arabidopsis events with enhanced tolerance to lethal drought were analyzed in a mild stress assay (Skirycz et al., 2011). The authors screened the literature in order to identify Arabidopsis genes that in gain- or loss-of-function situations confer drought stress tolerance without penalties in growth, and then selected 25 to perform the assay. In this assay, two lines showed larger plants while the rest were smaller, either in control or under drought conditions. However, growth reduction under mild stress was similar for all of the genotypes tested. The authors therefore concluded that enhanced survival under severe drought is not a good indicator for improved growth performance under mild/moderate stress conditions which can often be found in temperate climates. Superior survival under severe drought is often associated with constitutive activation of water-saving mechanisms, such as stomatal closure, that can lead to growth penalty.

Therefore, genes that are useful in conferring tolerance to severe stress conditions in transgenic plants and increase survival rates are in most cases detrimental to plant yield when the transgenic plant expressing such transgene(s) is exposed to mild stress conditions.

Among the 25 genes tested in the study Skirycz et al., 2011, several encode transcription factors (TFs), but none of them belong to the HD-Zip family.

One of the strategies to reduce losses in plant productivity is to increase natural stress tolerance, by strengthening endogenous systems. Transcription factors (TFs) play a crucial role in the plant response to environmental factors as well as in the morphogenetic program. They are proteins acting in trans, able to recognize and bind specific DNA sequences (cis-acting elements) localized in the regulatory regions of their target genes. When these proteins bind their targets, they activate or repress whole transduction signals pathways.

About 1500 TFs have been identified in plants using bioinformatics, and TFs comprise numerous gene families. However, while they might be involved in the response, they may not necessarily confer a tolerance. Therefore, it continues to be necessary to undertake a series of functional genomic experiments in order to test and demonstrate the effects of TFs on stress tolerance as such effects cannot be predicted (Arce et al., 2008).

HD-Zip proteins characterized by the presence of a homeodomain associated with a leucine zipper constitute one family of plant transcription factors. The association of the DNA binding domain (HD) with an adjacent dimerization motif (leucine zipper abbreviated ZipLZ or LZ) is a combination found only in the plant kingdom, although the domains are found independently of each other in a large number of eukaryotic transcription factors. This large family of plant TFs has been divided into four subfamilies (I to IV) according to sequence similarity in and outside the conserved domains and by the intron/exon patterns of the corresponding genes (Chan et al. 1998, Henriksson et al., 2005; Ariel et al., 2007). Members of subfamily I interact with the pseudopalindromic sequence CAAT(A/T)ATTG; subfamily II proteins recognize a motif CAAT(C/G)ATTG. In all cases, the formation of protein homo- or hetero-dimers is a prerequisite for DNA binding.

HaHB1 (Helianthus Annuus Homeobox 1) cDNA was isolated in 1992 from a sunflower stem cDNA library and its sequence was deposited in the Genebank (accession number L22847). HaHB1 is a member of the HD-Zip subfamily I (HD-Zip I). All HD-Zip I family members show high sequence similarity in the N-terminal homeodomain (HD) and leucine zipper domain (LZ), but are notably much more diverse in the C-terminal region. For example, HaHB1 and HaHB4 are both members of HD-Zip I, but they share very little sequence similarity in their C-terminal regions. Although HD-Zip I are grouped into a single family, different HD-Zip I family members exhibit differential expression patterns and are involved in different developmental and physiological processes. The disclosure in WO 2010/139993 and Cabello et al., 2012 (both incorporated herein by reference) demonstrate the utility of HaHB1 in the production of transgenic plants with enhanced tolerance to a number of different stresses under severe conditions. These disclosures also show that when exposed to different types of severe stress, such as drought, salinity, chilling or freezing, plants that have been transformed with a HaHB1 nucleic acid sequence and express a HaHB1 polypeptide show a higher survival rate compared to non-transformed control plants. In the disclosure in WO 2010/139993, the inventors have characterised HaHB1 and compared its structure to homologous sequences. Using chimeric constructs, the inventors have also found that it is the C-terminus of the HaHB1 protein that is important in conferring HaHB1 function. It was also shown that AtHB13 has a similar effect as HaHB1 when expressed in transgenic plants. Furthermore, other genes homologous to HaHB1 that show high homology not only in the HD and LZ domains, but also in the C-terminal domains, are also predicted to have a similar effect as HaHB1 when expressed in transgenic plants.

There is a need for methods for making plants with increased yield under moderate stress conditions. In other words, whilst plant research in making stress tolerant plants is often directed at identifying plants that show increased stress tolerance under severe conditions that will lead to death of a wild type plant, these plants do not perform well under moderate stress conditions and often show growth reduction which leads to unnecessary yield loss. The invention is aimed at addressing this need.

### SUMMARY OF THE INVENTION

Plant genes that are useful in conferring tolerance to severe stress conditions in transgenic plants and increase survival rates are in most cases detrimental to plant yield when the transgenic plant expressing such transgenes and grown under normal conditions is then exposed to mild stress conditions. The inventors have surprisingly found that expression of HaHB1 (which is known to confer stress tolerance under severe stress conditions) in transgenic plants results in plants that show increased yield compared to control plants when the plants are exposed to moderate stress or when the plants are well watered. In other words, the loss in yield experienced by a plant when exposed to moderate stress is less in plants expressing HaHB1 than in control plants. The presence of the transgene therefore mitigates against yield loss. Moreover, yield increases in the transgenic plants expressing HaHB1 compared to control plants have also been observed.

In the present patent disclosure, we describe the use of HaHB1, a transcription factor that is a member of the sunflower subfamily I of HD-Zip proteins and functional variants, functional parts or homologues thereof, such as AtHB13, to modify plant responses to moderate stress conditions, including freezing, drought, salinity and biotic stress. Thus, the various aspects of the invention all relate to uses of HaHB1, functional variants, functional parts or homologues thereof and methods which increase yield. Specifically, as shown herein these methods confer increased yield to a plant under moderate stress conditions. We demonstrate that by making transgenic plants expressing the sunflower HaHB1 cDNA under the control of the constitutive 35S promoter, transgenic plants are produced which exhibit clear increases in yield in response to moderate stress conditions compared to a control plant.

Thus, in a first aspect, the invention relates to a method for increasing yield of a plant under one or multiple moderate stress conditions comprising introducing and expressing in said plant a HaHB1 nucleic acid sequence, a functional part, homologue or functional variant thereof. The HaHB1 nucleic acid sequence may be as defined by SEQ ID NO:2, 6 or 7. In one embodiment, the HaHB1 nucleic acid sequence comprises or consists of SEQ ID NO:2, 6 or 7.

In another aspect, the invention relates to the use of a nucleic acid sequence as defined by SEQ ID NO:2, 6 or 7, a functional part, homologue or functional variant thereof in increasing yield under moderate stress conditions.

From the information provided herein, those skilled in the art will appreciate that HaHB1, a functional variant, functional part or a homolog thereof, confers enhanced yield under moderate stress conditions under the control of a constitutive promoter such as the CaMV 35S promoter, or under the control of another type of promoter, such as for example a cold inducible promoter or an abiotic stress inducible promoter or the HaHB1 promoter.

In light of the general information provided herein, those skilled in the art will appreciate that this invention describes and enables those skilled in the art to obtain and isolate a gene sequence from sunflower or related variants from other plant species which can be used to confer enhanced stress tolerance and thus increased yield to plants under moderate stress conditions, including those of chilling, freezing, tolerance, drought or high salinity and/or biotic stress.

### BRIEF DESCRIPTION OF THE DRAWINGS

FIG. 1. Sunflower HaHB1 and Arabidopsis AtHB13 were up-regulated by drought

Kinetics of expression of HaHB1 in sunflower R1 leaves (A) and AtHB13 in 21-day-old seedlings (B) subjected to drought stress during the periods indicated.

Transcript levels were quantified by qPCR and the values normalized with respect to that measured at time 0, arbitrarily assigned a value of one. Error bars are standard deviations calculated from three independent samples in which actin transcripts (ACTIN2 plus ACTING) were used as reference genes.

FIG. 2. Transgenic plants expressing HaHB1 or AtHB13 were tolerant to drought and exhibited stabilized membranes and higher yield in mild stress conditions

(A) Illustrative photograph of 25-day-old 35S:HaHB1 (H1-A, H1-B and H1-C), 35S:AtHB13 (A13-A, A13-B, A1-C) and WT plants grown in standard conditions and then subjected to drought for 17 days.

The experiment was carried out with 112 plants of each genotype and repeated three times. The photographs were taken 4 days after rehydration.

(B) Membrane stability (expressed as % of leakage) assay performed with 25-day-old transgenic (35S:HaHB1 and 35S:AtHB13 respectively) and WT plants subjected to drought stress for 17 days. (C) Chlorophyll content in 25-day-old plants under drought stress related to plant fresh weight. The samples were collected and quantified after 10 days of stress.

(D) Weight of seeds obtained after harvest of 35S:HaHB1 and WT plants grown in standard conditions or subjected to a mild stress throughout the life cycle as described herein. The assay was repeated three times and standard deviation was calculated from the results obtained from 4 plants of each genotype per experiment repetition. Asterisks indicate a significant difference between genotypes under the same condition with P<0.05 using ANOVA test.

FIG. 3. PR2 and PR4 were up-regulated by drought stress and the plants overexpressing these two genes or GLU exhibited stabilized membranes and higher chlorophyll concentration when subjected to drought

(A) Kinetics of expression of PR2 and PR4 in 21-day-old seedlings subjected to drought stress during the indicated periods.

Transcript levels were quantified by qPCR and the values normalized with respect to that measured at time 0, arbitrarily assigned a value of one. Error bars represent standard deviations calculated from three independent samples in which actin transcripts (ACTIN2 plus ACTIN8) were used as reference genes.

(B) Membrane stability assay performed with 25-day-old transgenic (35S:PR2, 35S:PR4 and 35S:GLU three independent lines for each transgene, named PR2 A, B, C; PR4 A, B, C and GLU A, B, C) and WT plants subjected to drought stress during 16 days.

(C) Chlorophyll content in 25-day-old plants under drought stress related to plant fresh weight. The samples were collected after 10 days of stress.

FIG. 4. Sunflower HaNB1 and Arabidopsis AtHB13, PR2, PR4 and GLU were up-regulated by salinity (NaCl) and the transgenic plants overexpressing these genes exhibited stabilized membranes under this stress

(A) Kinetics of expression of HaHB1 in sunflower R1 leaves and of AtHB13, PR2, PR3 and PR4 in 21-day-old seedlings subjected to salinity stress during the periods indicated as: a: one day after 50 mM NaCl addition; b: three days after 50 mM NaCl addition; c: one day after 150 mM NaCl addition; d: three days after 150 mM NaCl addition; e: one day after 200 mM NaCl addition; f: three days after 200 mM NaCl addition, and g: seven days after 200 mM NaCl addition.

Transcript levels were quantified by qPCR and the values normalized with respect to that measured at time 0, arbitrarily assigned a value of one. Error bars are standard deviations calculated from three independent samples in which actin transcripts (ACTIN2 plus ACTING) were used as reference genes.

(B) Membrane stability assay performed with 25-day-old transgenic (35S:HaHB1, 35S:AtHB13 35S:PR2, 35S:PR4 and 35S:GLU, three independent lines per genotype named H1-A, -B, -C; A13-A, -B, -C, PR2-A, -B, -C, PR4-A, -B, -C and GLU-A, -B, -C) and WT plants subjected to salinity stress during the periods described in (A).

(C) Weight of seeds obtained from 35S:HaHB1 (H1-A; H1-B; H1-C) or WT plants grown in standard conditions or subjected to salinity stress as described in the Methods section. The assay was repeated four times and standard deviation was calculated from the results obtained from 4 plants of each genotype per experiment. Asterisks indicate a significant difference between transgenic and WT plants under the same condition with P<0.05 using ANOVA test.

FIG. 5. PR2, PR4 and GLU are down-regulated in athb13 plants

RNA was extracted from 25-day-old athb13 mutant plants and transcript levels of the putative AtHB13 targets, PR2, PR4 and GLU were quantified by RT-qPCR. Average and standard deviation were calculated from three biological replicas.

FIG. 6. Plants transformed with PrH1:HaHB1 are tolerant to drought

(A) Illustrative photograph of 25-day-old PrH1:HaHB1 (lines A, B and C) and WT plants cultured in normal growth conditions and then subjected to drought over 17 days. The experiment was carried out with 16 plants of each genotype and repeated three times. The photograph was taken on the 15^{th} day.

(B) Weight of seeds obtained from PrH1:HaHB1 and WT plants grown in standard conditions or subjected to a mild stress as described herein. The assay was repeated three times and standard deviation was calculated from the results obtained from 4 plants of each genotype per experiment repetition.

### DETAILED DESCRIPTION OF THE PREFERRED EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

In a first aspect, the invention relates to a method for increasing yield of a plant under moderate stress conditions comprising introducing and expressing in said plant a HaHB1 nucleic acid sequence, a functional part, homologue or functional variant thereof. The HaHB1 nucleic acid sequence may be as defined by SEQ ID NO:2, 6 or 7. Thus, the sequence may comprise or consist of SEQ ID NO:2, 6 or 7.

The method therefore includes the preparation of a transgenic plant which expresses an HaHB1 sequence by a transformation method. Methods for plant transformation to introduce the HaHB1 transgene as described herein, for example by Agrobacterium mediated transformation or particle bombardment, and subsequent techniques for regeneration and selection of transformed plants are well known in the field. Also within the scope of the invention is chloroplast transformation through biobalistics.

The increase in yield in the transgenic plant is compared to a control plant which has not been transformed with a HaHB1 nucleic acid sequence to express said sequence, for example a wild type plant. The control plant is typically of the same plant species, preferably the same ecotype as the plant to be assessed.

As used herein, the words “nucleic acid”, “nucleic acid sequence”, “nucleotide”, or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in genomic sequence, or may comprise only a coding sequence as in cDNAs, and/or may include cDNAs in combination with regulatory sequences.

The term “stress” or “stress condition” as used herein includes abiotic and biotic stress. Said stress/stress tolerance is preferably selected from one or any combination of the following: freezing, low temperature, chilling, drought, high salinity and/or invasion of pathogens, for example Pseudomonas. In one preferred embodiment, the stress is drought. In another embodiment, the stress is high salinity. The inventors have shown that HaHB1 is able to trigger a variety of different stress pathways and can therefore confer increased tolerance to multiple stresses.

According to the methods of the invention, yield may be increased in response to one or more than one type of moderate stress. Therefore, in accordance with the various aspects of the invention, methods are provided that confer an increase in yield under not only a single, but more than one type of stress. In one embodiment, an increase in yield is conferred in response to all of the following stresses when said stress is moderate: freezing, low temperature, chilling, drought, high salinity and/or invasion of pathogens. Thus, the invention relates to a method for conferring increasing yield of a plant under multiple moderate stress conditions comprising introducing and expressing in said plant a HaHB1 nucleic acid sequence, a functional part, homologue or functional variant thereof wherein said stress is selected from at least two, three, four, five or all of freezing, low temperature, chilling, drought, high salinity and/or invasion of pathogens. Any combination is covered. For example, the stress may be drought and freezing and/or low temperature and/or chilling and/or high salinity and/or invasion of pathogens.

As shown herein, the transgenic plants show enhanced tolerance to these types of stresses compared to a control plant and are able to mitigate any loss in yield/growth. The tolerance can therefore be measured as an increase in yield as shown in the examples.

It is important to distinguish the difference between chilling and freezing conditions because the consequences and the responses triggered by the plants are not the same in these two distinct stresses. Prolonged freezing causes tissue death and, ultimately, plant death while prolonged chilling results in plant acclimation and developmental arrest.

Chilling and freezing tolerance occurs via different mechanisms. The response to chilling involves the activation of unsaturases which are able to change the lipid composition of the membranes generating increased membrane fluidity at low temperatures. On the other hand, freezing tolerance requires a previous acclimation period. During this acclimation period, certain specific proteins are synthesized and accumulated. Chilling and freezing are stresses that show different effects on plants: the first leads to slow biochemical reactions, such as enzyme and membrane transport activities; the second leads to ice crystal formation that can cause the disruption of the cell membrane system.

The terms moderate or mild stress/stress conditions are used interchangeably and refer to non-severe stress. In other words, moderate stress, unlike severe stress, does not lead to plant death. Under moderate, that is non-lethal, stress conditions, wild type plants are able to survive, but show a decrease in growth and seed production and prolonged moderate stress can also result in developmental arrest. The decrease can be at least 5%-50% or more. Tolerance to severe stress is measured as a percentage of survival, whereas moderate stress does not affect survival, but growth rates.

Accordingly, the term moderate stress as used herein results in a measurable decrease of growth rates in wild type plants. Assays that mimic moderate stress conditions for Arabidopsis thaliana plants are described herein and in Skirycz et al, 2011. The decrease may be at least 5%-50% or more, for example 5%-10%, 1-25%, 20-30%, 30-40%, 40-50%.

The precise conditions that define moderate stress vary from plant to plant and also between climate zones, but ultimately, these moderate conditions do not cause the plant to die. With regard to high salinity for example, most plants can tolerate and survive about 4 to 8 dS/m. Specifically, in rice, soil salinity beyond ECe ˜4 dS/m is considered moderate salinity while more than 8 dS/m becomes high. Similarly, pH 8.8-9.2 is considered as non-stress while 9.3-9.7 as moderate stress and equal or greater than 9.8 as higher stress.

Drought stress can be measured through leaf water potentials. Generally speaking, moderate drought stress is defined by a water potential of between −1 and −2 Mpa. This has for example been applied in experiments relating to barley and Phaseolus vulgaris L (Wingler et al, 1999 and Torres-Franklin et al 2007).

Moderate temperatures vary from plant to plant and specially between species. Normal temperature growth conditions for Arabidopsis are defined at 22-24° C. For example, at 28° C., Arabidopsis plants grow and survive, but show severe penalties because of “high” temperature stress associated with prolonged exposure to this temperature. However, the same temperature of 28° C. is optimal for sunflower, a species for which 22° C. or 38° C. causes mild, but not lethal stress. In other words, for each species and genotype, an optimal temperature range can be defined as well as a temperature range that induces mild stress or severe stress which leads to lethality.

Also, depending on the soil conditions and/or geographic region in which the plant is grown, “mild stress conditions” can be constant/permanent. For example, the yield of the same soybean (or maize) genotype exhibits differences every year when comparing different regions presenting varied rainfall regimes, even when no drought season occurred during this time.

Moderate stress conditions are common even in temperate climates and affect yield. A skilled person would be able to determine temperatures that can lead to mild stress for any given species based on common general knowledge in the technical field and/or routine methods.

The term “functional part or functional variant of HaHB1/HaHB1” as used herein refers to a variant gene or amino acid sequence or part of the gene or amino acid sequence which retains the biological function of the full non-variant sequence, i.e., it confers an increase in yield under moderate stress conditions when expressed in a transgenic plant. It also confers, as shown in WO 2010/139993, tolerance to severe stress and an increase in survival.

According to embodiments of the methods of the invention, a nucleic acid is expressed in a transgenic plant transformed with a vector as described herein or transformed with a gene sequence as described herein. Thus, the plant transformed according to the methods of the invention expresses or overexpresses a gene encoding for the sunflower HaHB1 protein or a functional variant or functional part thereof. Preferably, the HaHB1 protein comprises or substantially consists of a sequence as defined in SEQ ID NO:5, a functional part or functional variant thereof.

Specifically, the functional variant according to the methods of the invention may be a chimeric sequence that encodes for the C-terminus of HaHB1 as described herein wherein said C-terminus comprises or consists of SEQ ID NO:8. Alternatively, the chimeric sequence encodes a sequence comprising the CI and/or CII motif of HaHB1. In another embodiment, the chimeric sequence encodes a sequence comprising the CI and/or CII motif consensus sequences. These C-terminal sequences may be coupled to the N-terminus including the HD-Zip domain of another HD-Zip I family member.

The term “chimeric nucleic acid construct” is understood to mean that the two parts which are joined in the construct are originally derived from different nucleic acids and coded for separate proteins. Translation of this fusion gene results in a single polypeptide with functional properties derived from each of the original proteins.

The term “variant” may also refer to a sequence that encodes a peptide/protein sequence that is homologous to HaHB1 and which shows homology in the HD and LZ domains and also in the C-terminal domains (in particular to CI and CII). A functional variant also comprises a variant of HaHB1 which is substantially identical, i.e., has only slight sequence variations, for example in non conserved residues, to the HAaHB1 and HaHB1 sequences as shown herein and confers stress tolerance. A functional part may be the sequence encoding for the CI and/or CII motif.

The nucleic acids used in the methods of the invention may be part of an expression cassette that may, for example, be part of an expression vector. In an expression cassette or expression vector, the nucleic acid sequence is under the control of a promoter that preferably drives overexpression of the gene, such as the CaMV35S promoter. Other promoters that can be used are known to the skilled person. Also within the scope of the invention is the use of inducible promoters. Vectors may also have a selection marker to select transformed plants.

In one embodiment, the promoter is the endogenous promoter that directs expression of an HaHB1 gene. For example, the promoter may comprise or consist of the HaHB1 promoter as shown in SEQ ID NO:1 or a functional variant thereof. In one embodiment the promoter may comprise or consist of SEQ ID NO:1 directing the expression of a gene that is a functional homolog or variant of HaHB1.

The terms “regulatory element”, “control sequence” and “promoter” are all used interchangeably herein and are to be taken in a broad context to refer to regulatory nucleic acid sequences capable of effecting expression of the sequences to which they are ligated. The term “promoter” typically refers to a nucleic acid control sequence located upstream from the transcriptional start of a gene and which is involved in recognising and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e., upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. According to the invention, plants that express an HaHB1 nucleic acid sequence, or a functional variant of homolog thereof, show less decrease in yield than control plants when exposed to moderate stress. Yield is thus increased in plants that express an HaHB1 nucleic acid sequence compared to control plants. Yield can be measured in a number of ways, for example by measuring plant growth, seed production or biomass. An increase in yield can, for example, be assessed by the harvest index, i.e., the ratio of seed yield to aboveground dry weight. Thus, according to the invention, an increase in yield comprises one or more of: increased seed yield per plant, increased seed filling rate, increased number of filled seeds, increased harvest index, increased number of seed capsules/pods, increased seed size, increased growth or increased branching, for example inflorescences with more branches. Preferably, yield comprises an increased number of seed capsules/pods and/or increased branching. Yield is increased relative to control plants. The increase in yield is at least about 10%, 15%, 20%, 25%, 30%, 35%, 40%, 45% or 50% compared to a control plant. In one embodiment, the increase in yield is about 10% to about 50%.

Yield can also be measured as the difference in the loss of yield observed in plants that are grown under moderate stress conditions when compared to plants grown under normal conditions. As shown in the examples, both wild type plants and plants transformed with an HaHB1 transgene and expressing the HaHB1 protein have decreased yield when grown under moderate stress conditions when compared to plants grown under normal conditions. However, in plants expressing the HaHB1 protein, the decrease in yield is less than the decrease observed in the control plants. According to the methods described herein, the plant into which a vector or sequence as defined herein is introduced may be Arabidopsis. In WO 2010/139993, the inventors have shown that the HaHB1 gene sequence from sunflower can direct the expression of the HaHB1 protein in Arabidopsis. Moreover, the inventors have shown that transgenic expression of 35S:HaBH1 and expression of 35S:AtHB13 respectively in other plants or plant tissue results in the up-regulation of the expected target genes, thus providing evidence that both HaBH1 and ATHB13 are effective in exogenous plant hosts and that transgenic expression of HaBH1 and AtHB13 has universal application in genetically manipulating plants. The skilled person would thus know that the invention is not limited to Arabidopsis and that any monocot or dicot plant can be used according to the different aspects of the invention.

A dicot plant may be selected from the families including, but not limited to Asteraceae, Brassicaceae (eg Brassica napus); Chenopodiaceae, Cucurbitaceae, Leguminosae (Caesalpiniaceae, Aesalpiniaceae Arimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as sugar cane, oilseed rape/canola, linseed, and willow, poplar, poplar hybrids, switchgrass, Miscanthus or gymnosperms, such as loblolly pine. Also included are crops for silage (eg forage maize), grazing or fodder (grasses, clover, sanfoin, alfalfa), fibres (e.g., cotton, flax), building materials (e.g., pine, oak), pulping (e.g., poplar), feeder stocks for the chemical industry (e.g., high erucic acid oil seed rape, linseed) and for amenity purposes (e.g., turf grasses for golf courses), ornamentals for public and private gardens (e.g., snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, onion, leek, millet, buckwheat, turf grass, Italian rye grass, switchgrass, Miscanthus, sugarcane or Festuca species.

Preferably, the plant into which a sequence or vector of the invention is introduced is a crop plant. By crop plant is meant any plant which is grown on a commercial scale for human or animal consumption or use or other non-food/feed use.

Preferred plants are maize, tobacco, wheat, rice, oilseed rape, sorghum, soybean, potato, tomato, barley, pea, bean, field bean, cotton, lettuce, broccoli or other vegetable brassicas or poplar.

The gene or nucleic acid sequence used in the aspects of the invention may be an exogenous gene, such as sunflower HaHB1, a functional variant, part or homologue thereof, expressed in a different plant species, for example one of the plants listed above. For example, the homologue which is expressed or overexpressed as an exogenous transgene in a different plant, for example one of the plants listed above, may be a nucleic acid sequence comprising or consisting of SEQ ID NO:15 and encoding for Arabidopsis HaHB1 (AtHB13). Another example that can be used is ATHB23, a gene that shares high homology with HaHB1. Also included are functional variants and homologs of AtHB13 and AtHB23. Expression of AtHB13 or AtHB23 may be driven by the HaHB1 promoter comprising SEQ ID NO:1.

Alternatively, the invention also relates to using a plant's endogenous HaHB1 gene (which is endogenous to the plant in which it is introduced as a transgene by recombinant DNA technology and expressed or overexpressed) which is a homologue of the sunflower HaHB1, i.e., a gene encoding for a homologue of HaHB1.

The homologous gene shows high sequence similarity in the HD and LZ domains, but also in the C-terminal domains (CI and CII) as the C-terminus is crucial in conferring HaHB1 function.

A homologue of the sunflower HaHB1 polypeptide has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO:5. In one embodiment, a homologue of the sunflower HaHB1 polypeptide has at least 90%, 91%, 92%, 93%, 94%, 95%, 96%, 97%, 98%, or 99% overall sequence identity to the amino acid represented by SEQ ID NO:5. The overall sequence identity can be determined using a global alignment algorithm, such as the Needleman Wunsch algorithm in the program GAP (GCG Wisconsin Package, Accelrys), preferably with default parameters and preferably with sequences of mature proteins (i.e., without taking into account secretion signals or transit peptides).

A homologue of the sunflower HaHB1 nucleic acid sequence has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the amino acid represented by SEQ ID NO:2. In one embodiment, a homologue of the sunflower HaHB1 polypeptide has at least 90%, 91%, 92%, 93%, 94%, 95%, 96%, 97%, 98%, or 99% overall sequence identity to the amino acid represented by SEQ ID NO:2.

The classification of HD-Zip proteins into four subfamilies is supported by the following four distinguishing characteristics: conservation of the HD-Zip domain determining DNA binding specificities, gene structures, additional conserved motifs and functions (Ariel F D et al., 2007).

Members of the HD-Zip family exhibit a LZ motif just downstream from the HD motif. The two motifs are present in transcription factors belonging to other eukaryotic kingdoms, but their association with each other in a single protein is unique to plants. The HD is responsible for the specific binding to DNA while the LZ acts as a dimerization motif. HD-Zip proteins bind to DNA as dimers, and the absence of the LZ absolutely abolishes their binding ability, indicating that the relative orientation of the monomers, driven by this motif, is crucial for an efficient recognition of DNA (Ariel F D et al., 2007, Tron A E et al., 2005).

Thus, a homolog of the sunflower HaHB1 polypeptide is characterised by the presence of conserved domains, including the HD-Zip domain and the LZ motif.

In one embodiment the homolog has at least 90%, 91%, 92%, 93%, 94%, 95%, 96%, 97%, 98%, or 99% overall sequence identity to the amino acid represented by SEQ ID NO:8, 9, 10, 11 or 12.

In Arabidopsis, subfamily I is composed of seventeen members (ATHB1/HAT5, 3/HAT7, 5, 6, 7, 12, 13, 16, 20, 21, 22, 23, 40, 51, 52, 53, 54). HD-Zip I subsets of genes (in Arabidopsis) share their intron/exon distribution in accordance with their phylogenetic relationships. The molecular weight of the encoded proteins is about 35 kDa and they exhibit a highly conserved HD and a less conserved LZ. Other similarities and conserved motifs are described in Acre et al, 2011, incorporated herein by reference.

A full in vitro description, consisting of PCR-assisted binding site selection and footprinting assays, determined that all the proteins encoded by HD-Zip I genes and tested, recognize, as dimmers, the pseudopalindromic sequence CAAT(A/T)ATTG (Ariel et al., 2007).

WO 2010/139993 discloses consensus sequences, in particular for the carboxy terminus domain in Subfamily I. The C-terminal region provides a disordered protein-protein interaction domain, and the interaction of this domain with other diverse proteins determines the function of the protein. Without the HD-Zip domain, the HaHB1 C-terminus does not function autonomously. The HD-Zip of HaHB4 (with its C-terminus deleted) fused to the C-terminus of HaHB1, appears to behave like HaHB1. All the HD-Zip I members characterized up to now bind the same DNA sequence.

Therefore, it is believed that the HaHB1 C-terminus fused to the HD-Zip region of any subfamily I HD-Zip domain acts as a functional HaHB1. The C-terminal region confers protein function (Arce et al., 2011).

The methods and uses of the invention comprise the use of chimeric sequences/constructs which act as functional variants of HaHB1. For example, chimeric constructs which comprise the HaHB1 C-terminus fused to the HD-Zip region of any subfamily I HD-Zip domain act as a functional HaHB1 can therefore also be used in the methods for conferring increased yield as described herein.

In such a chimeric construct, the N-terminal region is characterised in that it comprises a homeodomain with homology to the consensus as shown in SEQ ID NO:14 (which was obtained from subfamily I), associated in its C-terminus to a leucine zipper. Moreover, this conserved homeodomain is able to bind the palindromic sequence CAAT(A/T)ATTG which is characteristic for this subfamily and differs from the sequences bound by members of other subfamilies.

Therefore, in one embodiment of the invention, the functional variant is a chimeric nucleic acid construct comprising a nucleic acid sequence encoding for the N-terminal sequence including the HD-Zip domain of an HD Zip protein of subfamily I or a sequence comprising a N-terminal consensus motif or part thereof operatively associated with a nucleic acid sequence encoding for a sequence comprising or consisting of the C-terminus of HaHB1, or a sequence comprising a C-terminal consensus motif or part thereof. The C-terminal sequence may comprise or consist of SEQ ID NO:8 or comprise one or more of SEQ ID NO:9, 10, 11 or 12.

Preferably, the C-terminal sequence comprises a sequence with at least 80%, preferably at least 90%, more preferably at least 95% homology to the consensus sequence of SEQ ID NO:8. In one embodiment, the homology is more preferably at least 95%, 96%, 97%, 98% or 99%.

In one embodiment, the N-terminal sequence including the HD-Zip domain of the chimeric construct comprises a sequence with homology to the consensus sequence of SEQ ID NO:14. Said homology may be at least 80%, preferably at least 90%, more preferably at least 95%. In one embodiment, the homology is more preferably at least 95%, 96%, 97%, 98% or 99%.

In another embodiment, the N-terminus including the HD-Zip domain of said chimeric polypeptide is the N-terminus of HaHB4 including the HD-Zip domain.

In another aspect, the invention relates to a method for producing a plant with increased yield under moderate stress conditions comprising introducing and expressing in said plant a nucleic acid sequence as defined herein.

The invention also relates to the use of a HaHB1 nucleic acid sequence, a functional part, homologue or functional variant thereof, preferably, a sequence comprising or consisting of SEQ ID NO:2, 6 or 7 to increase yield of a plant under moderate stress conditions.

A sequence or vector described herein encoding for the HaHB1 protein is introduced as a transgene into the plant.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors according to the invention, all those constructions brought about by recombinant methods in which either

(a) the nucleic acid sequences encoding proteins useful in the methods of the invention, or
(b) genetic control sequence(s) which is operably linked with the nucleic acid sequence according to the invention, for example a promoter, or
(c) a) and b)
are not located in their natural genetic environment or have been modified by recombinant methods. The natural genetic environment is understood as meaning the natural genomic or chromosomal locus in the original plant. The environment flanks the nucleic acid sequence at least on one side and has a sequence length of at least 50 bp, preferably at least 500 bp, especially preferably at least 1000 bp, most preferably at least 5000 bp.

A transgenic plant for the purposes of the invention is thus understood as meaning, as above, that the nucleic acids used in the method of the invention are not at their natural locus in the genome of said plant, it being possible for the nucleic acids to be expressed homologously or heterologously.

Methods that solely rely on conventional breeding techniques and do not involve recombinant technologies are disclaimed.

It will be understood by the skilled person that the transgene is preferably stably integrated into the transgenic plants described herein and passed on to successive generations.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### EXAMPLES

Having generally described the invention disclosed herein, including methods by which those skilled in the art could make and use the invention, the following examples are provided to further extend this description, to enable those skilled in the art to practice this invention, including its best mode. However, the specifics of the examples which follow are not limiting. Rather, for an appreciation of the scope of the invention contemplated herein, reference should be had to the appended claims and the equivalents thereof.

### Materials and Methods

### Constructs and Transgenic Plants

35S:HaHB1, 35S:AtHB13, 35S:PR2, 35S:PR4, ProH1:HaHB1, 35S:GLU and 35S:GUS constructs (used as control) as well as the transgenic plants bearing these constructs were previously described (Cabello et al., 2011). AtHB13 mutant plants (athb13 background) were obtained from the ABRC (Arabidopsis Biologic Resource Center) as heterozygous and homozygous lines were selected after two complete life cycles.

### Plant Material and Growth Conditions

Arabidopsis thaliana Heyhn. ecotype Columbia (Col-0) was purchased from Lehle Seeds (Tucson, Ariz.) and reproduced in the culture chamber. WT and transgenic plants were grown directly in soil in a growth chamber at 22-24° C. under long-day photoperiods (16 h of illumination with a mixture of cool-white and GroLux fluorescent lamps) at an intensity of approximately 150 μE m⁻¹ s⁻¹ in 8 cm diameter×7 cm height pots during the periods of time indicated in the figures.

Helianthus annuus seeds were germinated on wet filter paper for 7 days and then transferred to 8×7 cm pots each with an equal amount of vermiculite-perlite mix, one plant per pot and water-saturated. They were placed in a 45-cm plastic square tray and cultured as described below except that further water was not added until stress was evident. Plants were harvested and frozen for RNA isolation.

### RNA Isolation and Expression Analyses by Real Time RT-PCR

RNA for real-time RT-PCR was prepared with Trizol® reagent (Invitrogen™) according to the manufacturer's instructions. RNA (2 μg) was used for the RT reactions using M-MLV reverse transcriptase (Promega). Quantitative PCRs were carried out using a MJ-Cromos 4 apparatus in a 20 μl final volume containing 1 μl SyBr green (10×), 8 pmol of each primer, 2 mM MgCl₂, 10 μl of a 1/25 dilution of the RT reaction and 0.12 μl Platinum Taq (Invitrogen Inc.). Fluorescence was measured at 78-80° C. during 40 cycles. Sunflower RNA was also prepared with the Trizol (Invitrogen Inc.) technique, but in this case the dilution of the RT reaction was 1/50.

Specific oligonucleotides for each gene were designed using publicly available sequences (Arabidopsis.org web page and ncbi.nlm.nih.gov). The designed sequences specified below, correspond to the oligonucleotides used for qRT-PCR.

H1qF:
(SEQ ID NO: 17)
5′ GGCCGGCAGATCATCAACTTC 3′
H1qR:
(SEQ ID NO: 18)
5′ CCAACCATGGCCAAAACCCTG 3′
A13qF:
(SEQ ID NO: 19)
5′ CTCCATGGATTTGCTTCG 3′
A13qR:
(SEQ ID NO: 20)
5′ TCCCATTTGTGACCCATC 3′
qAtPR2-F:
(SEQ ID NO: 21)
5′ TAAGAAGGAACCAACGTATGAGAA 3′
qAtPR2-R:
(SEQ ID NO: 22)
5′ CATAAAAAGCCCACAAGTCTCTAA 3′
qAtPR4-F:
(SEQ ID NO: 23)
5′ GTTTAAGGGTGAAGAACACAAGAAC 3′
qAtPR4-R:
(SEQ ID NO: 24)
5′ ATTGAACATTGCTACATCCAAATC 3′
qAtGLU-F:
(SEQ ID NO: 25)
5′ ACCAAAACCTCTTTGACGCTTTAC 3′
qAtGLU-R:
(SEQ ID NO: 26)
5′ AATACGTTTCCACTCCTCTTCCAG 3′

### Water Stress and Salinity Treatments

Early water-stress treatment in soil was carried out as follows: 16 pots (8×7 cm), each one with a weighed amount of vermiculite-perlite mix was used to germinate 4 seeds, water-saturated and placed in a 45-cm plastic square tray. For severe drought assays, plants were not watered again until damage was observed. During this period, samples were harvested for RNA or cell membrane tests.

Water-stress treatment was also carried out with standard-conditions using 4-week-old plants. At this point, watering was stopped until damage became evident (approximately 16 additional days). In both cases, photographs were taken 2 days after re-watering.

Mild water stress treatments were carried out as follows: Plants were grown in standard-conditions for 25 days. Then, watering was stopped until a mild stress level was reached, 10 days later. Subsequently, the stress was maintained by watering the pots every two days, maintaining the same weight in all the pots, equal to the weight measured on day 35.

Salinity stress was performed as follows: 1 l (one litre) of 50 mM NaCl was added to the plastic square tray in which 16 pots with 25-day-old plants were placed. After 7 days another 1 l of 150 mM NaCl was added. Fourteen days after the first addition an additional 1 l of 200 mM NaCl was added. The samples for RNA extraction were collected one and three days after each NaCl addition and for membrane stability assay one day after each NaCl addition.

For seed production experiments, salinity stress was performed as follows: 1 l of 100 mM NaCl was added to the plastic tray with 16 pots containing each one a 21-day-old plant. After 7 days another 1 l of 100 mM NaCl was added. Fourteen days after the first addition an additional 1 l of 100 mM NaCl was added. After that, the plants were watered normally until harvesting.

### Cell Membrane Stability Evaluation by the Ion Leakage Technique

The ion leakage technique was carried out essentially as described by Sukumaran and Waiser (1972) with minor changes. Under drought, leaves were detached at different times as described in the figure legends. Under salinity stress, leaves were detached three days after each NaCl addition.

Leaves were exhaustively washed with distilled water prior to subjecting them to the stress by placing individual leaf samples in 15 ml of double-distilled deionized water with continuous agitation in a water bath at 25° C. for 3 hr. After decantation the aqueous extract conductance (C1) was measured. Then, the leaves were placed in a 65° C. water bath for 16 h with continuous agitation and one additional hour at 25° C. prior to measure the solution conductance (C2). The real conductance was calculated as the ratio between C2/C1 (L=C1/C) and used as index of injury. L values higher than 0.5 indicate severe injury.

### Chlorophyll Quantification

Chlorophyll was quantified following the method described by Chory et al., 1994. Hundred mg of rosette leaves from 25-day-old plants were weighted and pulverized using liquid N₂. The powder was transferred to a 1.5 ml tube and 1.5 ml of 80% acetone was added. The tubes were placed in the dark for 30 minutes and then centrifuged 5 minutes at 12000 rpm. Absorbance at 645 and 663 nm was measured in the supernatants. Total chlorophyll content was calculated using the following formula: Total chlorophyll (μg/ml)=20.2*A₆₄₅+8.02*A₆₆₃

TABLE 1
Survival rate of overexpressing lines for each of
HahB1, AtHB13, PR2, PR4 and GLU
A total of three overexpressing lines for each genotype with 64
(for WT, A13, PR2, PR4 and GLU) or 112 (for WT or H1) plants per
line were assayed under severe drought. Data represents three
experimental repetitions, and it is reported as the percentage
of average survival rate with the standard error.

| | | Average No of | | |
|---|---|---|---|---|
| | Genotype | survivors (%) | SE | N |
| 35S: HaHB1 | WT | 19 | 7 | 112 |
| | H1-A | 94 | 9 | 112 |
| | H1-B | 81 | 13 | 112 |
| | H1-C | 78 | 16 | 112 |
| 35S: AtHB13 | WT | 13 | 11 | 64 |
| | A13-A | 86 | 6 | 64 |
| | A13-B | 85 | 11 | 64 |
| | A13-C | 71 | 14 | 64 |
| 35S: PR2 | WT | 31 | 17 | 64 |
| | PR2-A | 72 | 13 | 64 |
| | PR2-B | 50 | 8 | 64 |
| | PR2-C | 60 | 13 | 64 |
| 35S: PR4 | WT | 22 | 8 | 64 |
| | PR4-A | 73 | 21 | 64 |
| | PR4-B | 80 | 24 | 64 |
| | PR4-C | 65 | 16 | 64 |
| 35S: GLU | WT | 29 | 7 | 64 |
| | GLU-A | 75 | 21 | 64 |
| | GLU-B | 83 | 20 | 64 |
| | GLU-C | 79 | 10 | 64 |

### Results

### HaHB1 and AtHB13 are Up-Regulated by Drought

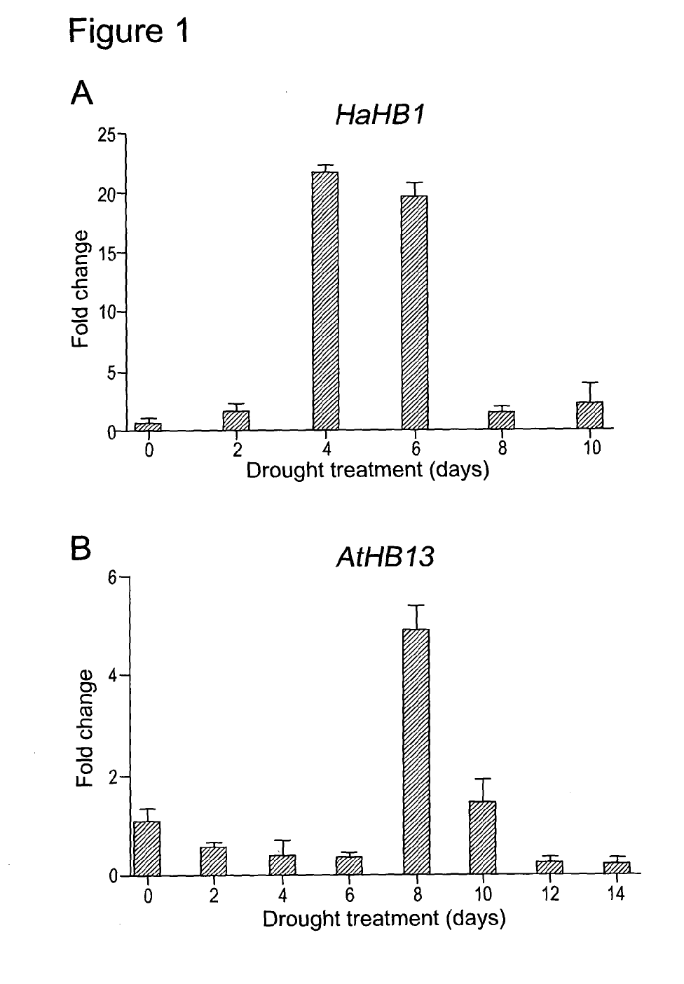

In order to characterize in detail the expression of HaHB1 and AtHB13 in conditions of abiotic stresses, total RNA was isolated at various time points from R2 Helianthus annuus leaves and from 25-day-old WT Arabidopsis rosette leaves, both subjected to drought stress for 10 and 14 days respectively. Transcript levels of each gene were determined by qPCR with specific oligonucleotides. FIG. 1 shows that HaHB1 was induced around 20-fold after 4 days of starting the stress treatment while AtHB13 around 5-fold after 8 days, both slowly decreasing after this period. These results suggested a possible involvement of these genes in the drought response.

Transgenic Plants Ectopically Expressing HaHB1 or AtHB13 Exhibited Tolerance of Drought and Increased Yield Under this Stress

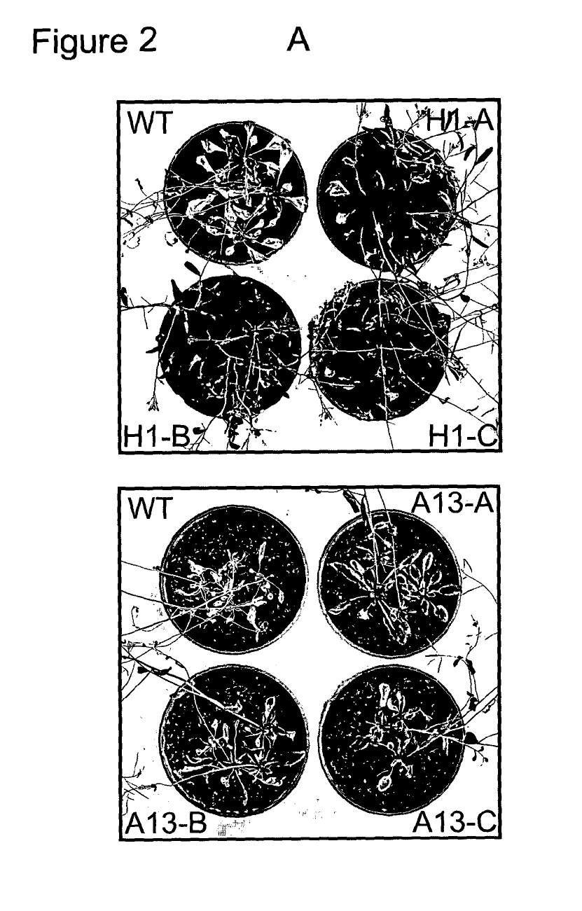

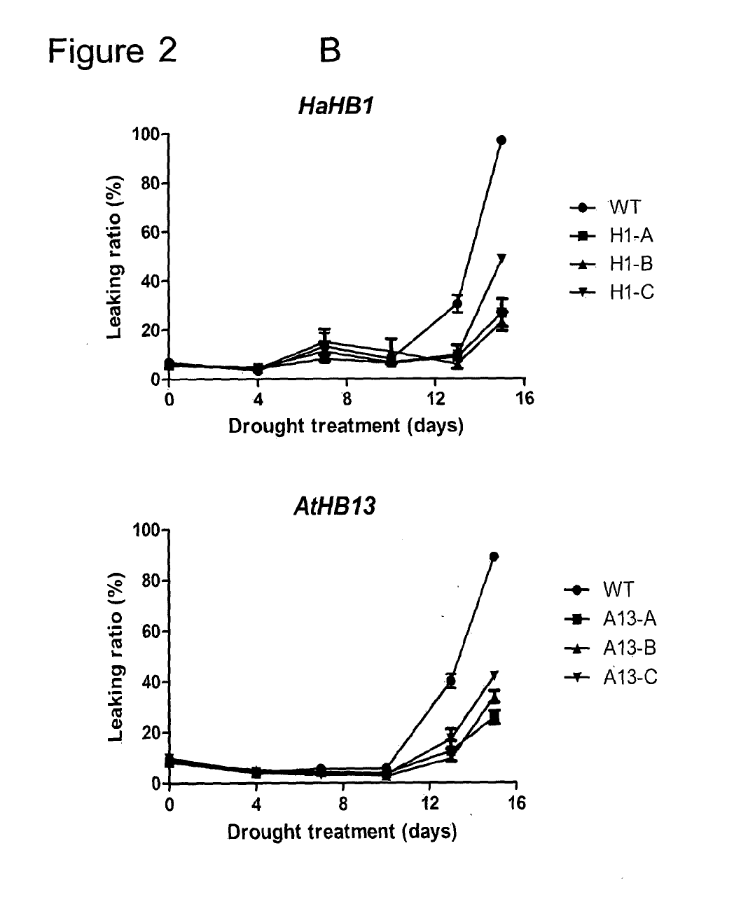

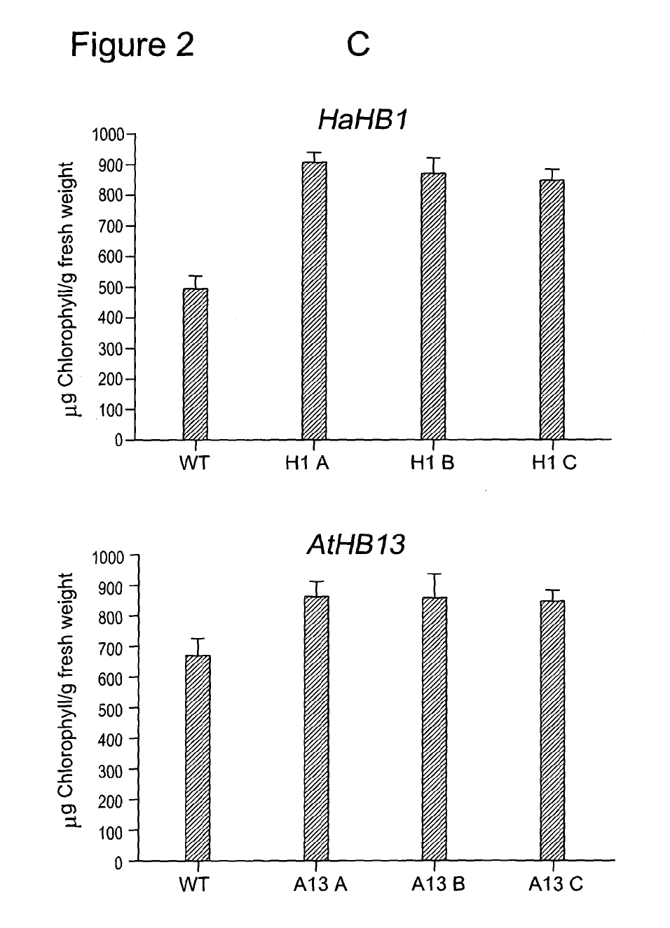

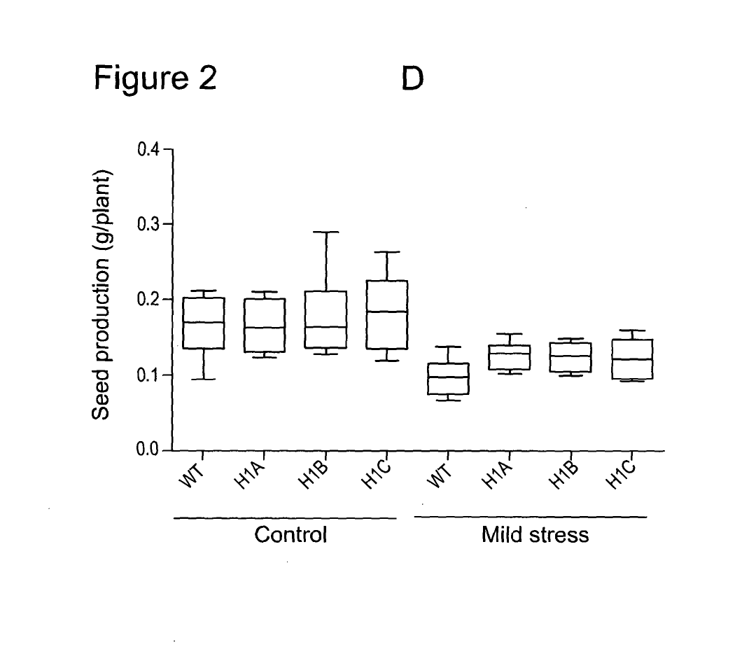

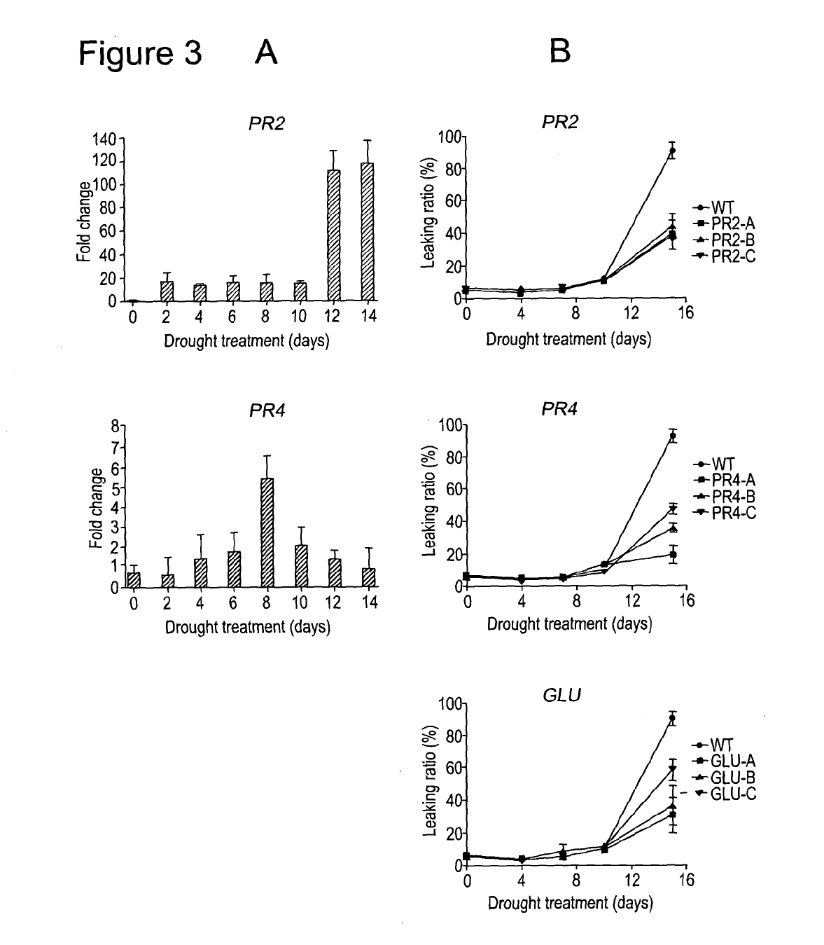

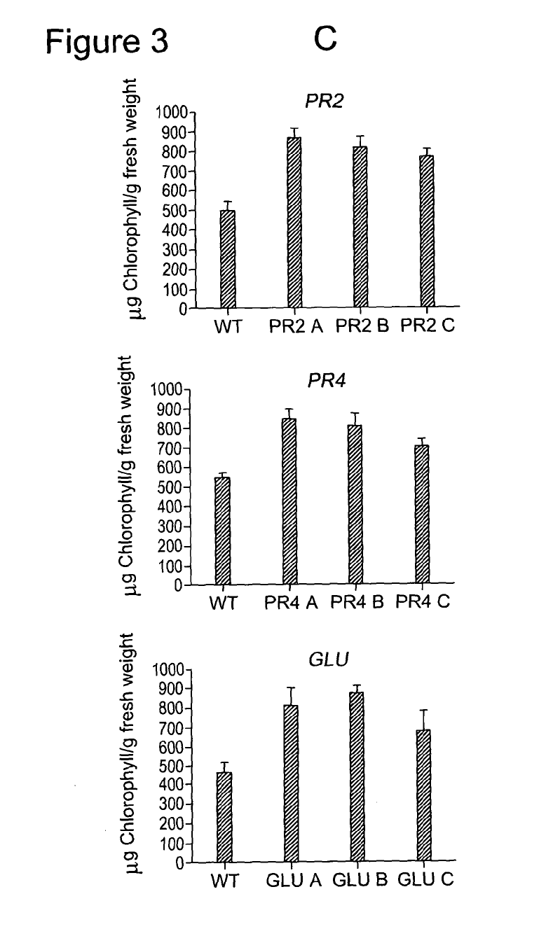

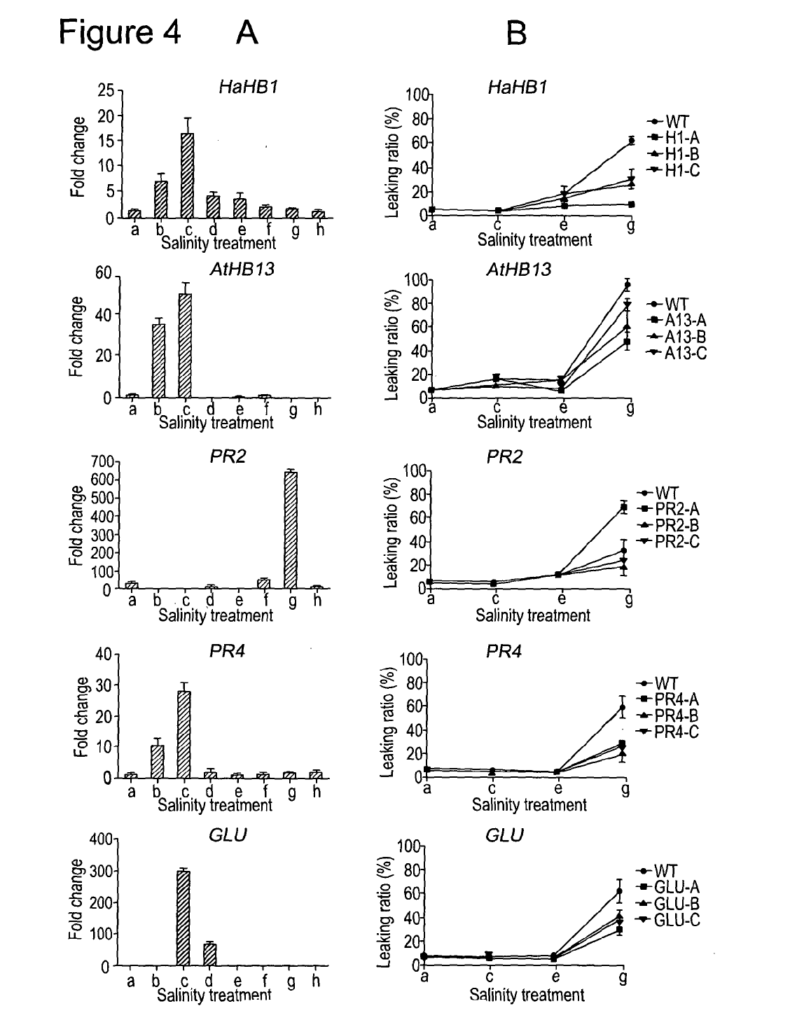

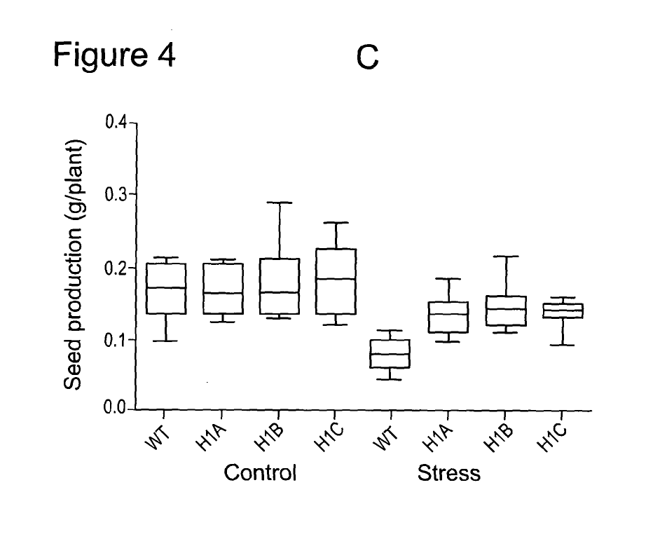

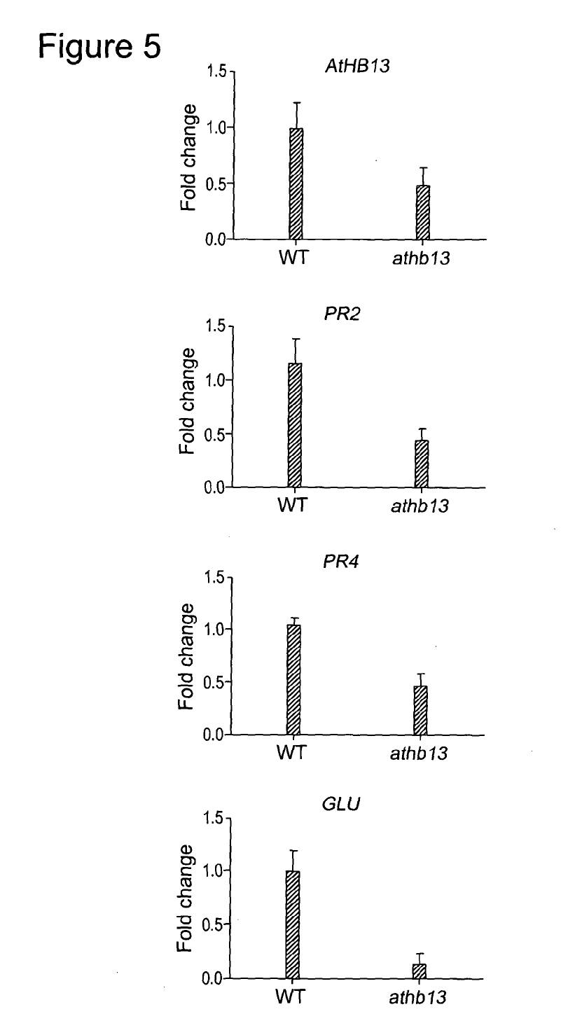

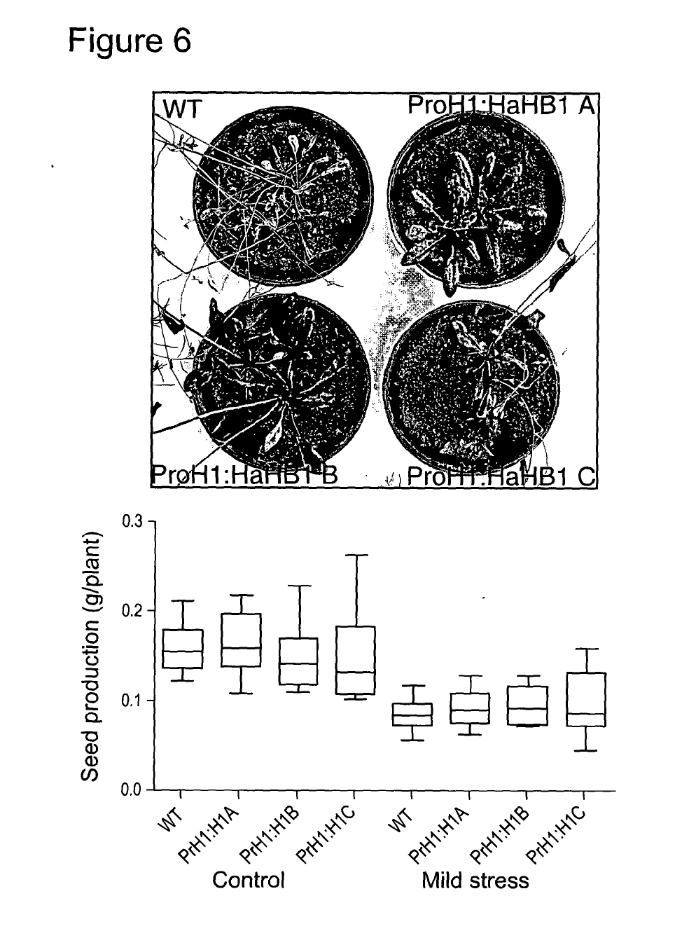

Considering the known up-regulation of HaHB1 and AtHB13 by drought, Arabidopsis plants transformed with these genes under the control of the 35S CaMV promoter were tested to determine their phenotype under this stress. Plants transformed with the pBI 101.3 plasmid (thereafter called WT) were used as controls. Individuals from three independent lines of each genotype were grown in standard conditions during 4 weeks and then no water was applied during the following 16 days; this treatment gradually resulted in a severe drought condition. After that, the plants were watered and allowed to recover over a period of two days. A significant difference in the percentage of survivors was observed between transgenics and WT (Table 1). FIG. 2a shows an illustrative image of HaHB1 and AtHB13 plants after the treatment. The experiment was repeated several times with plants either in the vegetative or in the reproductive stage with similar results.

The membrane state of these plants was evaluated during the treatment by the ion leakage technique and the results indicated that transgenic plants had a better cell membrane stabilizing mechanism than their WT counterparts, especially during the last/severe stress period (FIG. 2b). This difference could be at least in part the cause of the higher survival rate of the transgenics following the stress treatment. Chlorophyll content was also quantified as an indication of plant health and stress-provoked senescence. HaHB1 and AtHB13 plants exhibited a higher chlorophyll concentration during the stress treatment than the WT controls consistent with their more stabilized membranes (FIG. 2c).

Considering that while tolerance of a lethal stress has value as a biotechnological indicator, less extreme stress conditions are more widespread in agriculture and so, plants were subjected to mild stress conditions, in order to emulate these. Four week-old plants grown in standard conditions were either given a normal watering regime or subjected to mild stress by stopping the watering for a 10-day period. After that, the pot weight was maintained constant by weighting each pot daily and then adding water to compensate for any loss (see Experimental procedures). This mild water stress treatment did not cause plant death and plants flowered and set seeds, so that seed productivity could be measured for each plant. FIG. 2d shows the seed weight obtained for HaHB1 and WT plants in standard conditions or under mild stress. When the plants were well watered, the transgenics and WT both produced a similar seed weight of around 160-180 mg/plant. Both genotypes decreased in yield after a continuous mild stress; however, the difference in yield between treatments observed for WT plants was significantly larger (−43%) than for transgenics (−30% average).

Arabidopsis PR2 and PR4 were Up-Regulated by Drought and the Transgenic Plants Ectopically Expressing these Genes and GLU Exhibited Tolerance of Drought

The exploratory transcriptome analysis performed with plants transformed with 35S:HaHB1 indicated that genes encoding glucanases and chitinases proteins were upregulated (Cabello et al., 2011). These plants as well as plants expressing the homologous AtHB13 or the indirect targets PR2, PR4 and GLU, exhibited tolerance of freezing temperatures and displayed stabilized membranes after such stress. Wondering if the HD-Zip TF target genes were also regulated by drought stress, total RNA was isolated from 25-day-old Arabidopsis plants subjected to severe drought (watering was completely stopped) for a 14-day period. FIG. 3 shows that PR2 transcripts strongly increased from the 12^{th} day continuing until the 14th. After that, the plants were too severely damaged to extract RNA. PR4 was induced after 8 days of treatment decreasing after that, while GLU expression could not be detected in Arabidopsis leaves either after this stress treatment, probably due to its specific expression in roots (FIG. 3a).

Considering this expression pattern, transgenic Arabidopsis plants expressing PR2, PR4 and GLU (all them under the control of the 35S CaMV) were tested in conditions of drought stress. Individuals from three independent lines of each genotype were grown in standard watering conditions for 25 days and then no water was applied for the next 16 days, which resulted in a potentially lethal drought condition. After this treatment, the plants were watered, placed in the culture chamber to recover (or not) and the survivors were counted. A strong difference between transgenics and WT was observed (Table 1).

Notably, GLU plants exhibited tolerance in spite of the fact that, as mentioned above, transcripts of this gene could not be detected in stressed WT leaves. The experiment was repeated several times, both in the vegetative and in the reproductive stage with similar results. Membrane stability was evaluated during the treatment and the results, shown in FIG. 3b, indicated that transgenics triggered a better cell membrane stabilizing mechanism than their WT counterparts. This difference could be at least in part the cause of tolerance. Moreover, the chlorophyll content of these plants was quantified during drought stress. The results, shown in FIG. 3c, indicated that transgenic plants had more chlorophyll than their controls.

HaHB1, AtHB13, PR2, PR4 and GLU were Up-Regulated by Salinity Stress and Transgenic Plants Overexpressing these Genes Exhibited More Stabilized Cell Membranes and Increased Yield

Since separate signal transduction pathways activated to deal with different abiotic stresses are usually connected at some point or other, we wondered if HaHB1, AtHB13 and their target genes were, besides their participation in cold and drought responses, also involved in the response to the stress caused by salinity. HaHB1, AtHB13, PR2, PR4 and GLU transcript levels were quantified in plants subjected to salt stress. Total RNA was isolated at different periods of time from R2 sunflower leaves and from 25-day-old WT Arabidopsis plants as described in Experimental procedures. FIG. 4 shows that HaHB1, AtHB13, PR4 and GLU transcripts increase considerably three days after the addition of 50 mM NaCl while PR2 transcripts start to increase later and after a 200 mM NaCl addition.

The relative increase in expression was slightly different for each gene and also was dependent on the detectable basal expression levels (almost undetectable for GLU and PR2). Cell membrane stability was also evaluated under salinity stress for all the transgenic genotypes described above and the results shown in FIG. 4. Like under the other abiotic stress factors tested, the transgenic genotypes displayed a better response under high salt concentrations suggesting that these plants might have higher tolerance of salinity than WT plants. However, this enhanced cell membrane stability was not sufficient to confer the tolerance needed to cause a difference in survival rate, at least under these particular high salt conditions.

In order to test seed production after a salinity stress, two week-old plants grown in standard conditions were treated with NaCl reaching a final 300 mM concentration (see Experimental procedures) and after that, watered normally. This treatment did not cause plant death and seeds could be individually collected. FIG. 4c shows that both genotypes (HaHB1 and WT) produced a similar yield of around 160-180 mg/plant in control conditions and both decreased under stress. However, the difference between treatments for WT was significantly larger (−50%); than for transgenics (−20% average).

PR2, PR4 and GLU were Down-Regulated in Athb13 Mutant Plants Indicating that these Proteins are Targets of AtHB13

Athb13 mutants (athb13) were obtained from ABRC seed stock. Homozygous lines were selected after kanamycin-resistance selection, then reproduced and grown under normal conditions. RNA was extracted from 25-day-old-rosette leaves and RT-qPCR analysis indicated that PR2, PR4 and GLU were down-regulated in athb13 plants in comparison with WT plants.

Transgenic Plants Expressing HaHB1 Under the Control of its Own Promoter were Tolerant to Drought Conditions

A 1021 by HaHB1 promoter region was previously isolated (Cabello et al., 2011) and found to contain cis elements such as ABRE and DRE, characterized as responsive to abiotic stresses. When Arabidopsis transgenic plants transformed with the construct PrH1:HaHB1 were subjected to drought and salinity stresses, they displayed a better phenotype than their controls (FIG. 5 and Table 1) indicating that the HaHB1 promoter was able to sufficiently induce HaHB1 expression, which in place triggered drought/salinity tolerance. In order to evaluate the yield of these plants either in normal or mild stress conditions, a productivity assay was performed. No differences between genotypes were detected (FIG. 6b) indicating that this construct was less efficient in providing tolerance than the constitutive 35S:HaHB1

### REFERENCES

Arce A. L., Cabello J. V., Chan R. L. 2008. Patents on Plant Transcription Factors. Recent Patents on Biotechnology, 2: 209-217
Arce, A. L., Raineri, J., Capella, M., Cabello, J. V. and Chan, R. L. (2011) Uncharacterized conserved motifs outside the HD-Zip domain in HD-Zip subfamily I transcription factors; a potential source of functional diversity. BMC Plant Biol. 11, 42.
Ariel F. D., Manavella P. A., Dezar C. A., Chan R. L. 2007. The true story of the HD-Zip family. Trends Plant Sci. 12, 419-426.
Cabello, J. V., Arce, A. L. and Chan, R. L. (2012) The homologous HD-Zip I transcription factors HaHB1 and AtHB13 confer cold tolerance via the induction of pathogenesis related and glucanase proteins. Plant J. 69, 141-153
Chan R. L., Gago G. M., Palena C. M., Gonzalez D. H. 1998. Homeoboxes in plant development. Biochim. Biophys. Acta 1442: 1-19.
Chory, J., Reinecke, D., Sim, S., Washburn, T. and Brenner, M. (1994) A role for citokinins in de-etiolation in Arabidopsis. Plant Physiol. 104, 339-347.
Henriksson, E., Olsson, A. S. B., Johannesson, H., Johansson, H., Hanson, J., Engstrom, P. and Soderman, E. (2005) Homeodomain Leucine Zipper Class I Genes in Arabidopsis. Expression Patterns and Phylogenetic Relationships. Plant Physiol. 139, 509-518.
Skirycz, A et al (2011) Survival and growth of Arabidopsis plants given limited water are not equal. Nat. Biotechnol. 29, 212-214.
Torres-Franklin et al (2007) Molecular cloning of glutathione reductase cDNAs and analysis of G R gene expression in cowpea and common bean leaves during recovery from moderate drought stress Journal of Plant Physiology Volume 165, Issue 5, 31 Mar. 2008, pages 514-521
Tron A E, Comelli R N, Gonzalez D H: Structure of homeodomain-leucine zipper/DNA complexes studied using hydroxyl radical cleavage of DNA and methylation interference. Biochemistry 2005, 44:16796-16803
Wingler et al (1999) The role of photorespiration during drought stress: an analysis utilizing barley mutants with reduced activities of photorespiratory enzymes Plant, Cell and Environment 22, 361-373

### Selected Sequences

HAHB1 promoter
SEQ ID NO: 1
gtcgagctcgtctcgtaaaatgttcgagtcagctccaattaaatcat
gtcggcttgttatattttttttaatttatttttgatattttttacat
atatttataacataaaaaacaaaataaaaataaaaattacacatata
tctatatgtattatttttctaaaattttaaatagcgaaagacatatt
aaaagtattatatgtataattttgtttagcttcccatatttttatat
gttattaattaattaaacttaaaatgttaacactttaacacctctta
catactttttagttcaatacatttaaaattaaaattaatctatatgc
aataaaataattcaagcaggcttgcaagctcacgagtcgagccatgc
ctggctcgagctcgactcatttacaaatcgagccaccaagccgactc
gtttataaccgagttttttttagccgagtttttttcaagcgaacttc
aaacaagtcacgcgcttttaattaacacattctagtctaaagaagat
aattgaaagagaaagtagatataagtaaaaggagtagccaaagatat
aaatttagggtctaaacaacctaatattgtttaattttttttaaata
aactagtttttttttaccgattatctgtgttatatgtcttagtttga
catgataagttatcataattacttgtagtatttttatatcagaaata
tacgttggagaattaaattttatcctgatcgtcaattgacaagaaca
aaaatcaacatctcatggttttttactaatttatatgattaaagata
tatggttgtaagaaaaagaacaatgtacatcaaatggtgaaatttga
atatttgatagtaacgtaatccattgtgtatttcttattattttatc
attttcccaaggtgtgtcatatatagtgtctccattctttctatagc
acaatatccttcacctccctctctctctctctctctctaaaaatgat
gatgagacgacaaagatcgaattc
HaHB1 mRNA
SEQ ID NO: 2
uugaaauucugagaaaagccacauaaucaaagcuaaagaggugguuu
aaacagcugAUGACUUGCACUGGAAUGGCUUUCUUCUCCUCCAAUUU
CAUGUUACAAUCCUCCCAAGAAGAUGACCAUCAUGCCCCUACAUCUC
UCUCUCCAAUCCUCCCACCUUGCAGUACCACCACUCAAGAUUUCAGU
GGUGCUGCUUUCUUGGGAAAAAGAUCUAUGUCUUCUUACUCAGGUUU
GAACAACAACAACAUGGAUGGAUGUGAUCAAGAAGGGAACAUGAAUG
GAGAAGAUGAGUUAUCAGAUGAUGGAUCACAGCUUCUUGCAGGAGAG
AAAAAGAGGAGAUUAAACAUGGAACAAGUGAAGACACUUGAGAGAAA
CUUUGAGUUAGGAAAUAAGCUUGAACCUGAGAGGAAAAUGCAACUUG
CAAGAGCACUUGGACUACAACCAAGACAGAUUGCUAUAUGGUUUCAA
AACAGAAGAGCUAGAUGGAAAACUAAACAGUUGGAAAAAGACUAUGA
UGCCCUCAAGAGACAGUUUGAAGCUGUUAAAGCUGAGAAUGAUUCAC
UCCAAUCUCAAAAUCAUAAACUUCAUGCUGAGAUAAUGGCACUAAAA
AAUAGGGAGCCAGCAGAACUAAUCAACCUCAACAUAAAAGAAACAGA
AGGAUCUUGCAGCAACCGAAGCGAAAACAGCUCUGAAAUCAAACUAG
ACAUCUCAAGAACACCGGCUACCGAUAGCCCUUUAUCAUCACACCAU
CAACACCAACACCAGCCAAUACCUAAUCUUUUUCCAUCGUCGAAUAU
CGAUAGGCCUAAUUCGAAUAACAUUGUGGCGCAUCAACUUUUCCACA
AUUCGUCAUCAAGGCCGGCAGAUCAUCAACUUCAUUGCCACAAACUC
GAUCAAUCGAAUGCCAUUAAAGAAGAAUGUUUUAGCACAAUGUUUGU
UGGUAUGGAUGAUCAAUCAGGGUUUUGGCCAUGGUUGGAACAACCAC
AAUUCAAUUGAuggaaucaagaagcaaaaaagcaaaagaaaacggua
cccgauucgccuucuuggcuuugguuugauuauauuaaagauggaga
ucaucaaucuguuuguucucuaagcuuuaaauucuuguuuuuuggua
cuuaaauuaauagaguaaaaauuagaagaaaaaacguauuauuauuu
uaaauucaagauuaguguuu

Coding sequence: in upper case
5″ and 3′ untranslated regions: in lower case
Flanking nucleotides from first and second introns: in bold and underlined

Intron 1
SEQ ID NO: 3
aattaactcaccttaactaagttacttatgacaacatctctctcata
gatcttgatgcagcttgcattcatgagttgtgatgtacaactcattc
atgcattagggtttcagttttttcaaagttttttttttattttttct
tctgtttcaagatcatgatgatgagttgtgctgaacacttgaacagc
tcattgatgcattagggtttgttttagtttcaagttctttcttttct
ttcattttcatgcactaaatccatatgggcttgaagaaagtttgaat
ctttatatgttagttgatgatcttgatgcaggt
Intron 2
SEQ ID NO: 4
gtaatattagtttgattgtttattgcatctatcaatcattagattct
actctttacttgatcacacagaaagtaactaaaccttttttcctaat
gataacaatatttgttttgcaaatctaatggcaatcaaataaaagtt
tctggtaagcagccatgatctatttatttttcactatttgagtaagt
ttaaaagttgcatttatcctcactaattatatacaacactaaaataa
tcattaaactgactgttataattactttccgtaaacggtatgccaaa
acttaaaatgattaacaattttataagaatggaaagtaaaatcatta
cactatttcccatattagtcatgaccaaagtttgtttctttctgaag
ggcaaaagggtcaatatgcttatatgcagcatgggcaaaagaagtag
agtgtatatcaaaattcatatctttattttcttttcaaagtttaggt
aacaaaaagaagaaattataaacgagtttgttacaattccacaagta
catgaagaaacaaaatttgttagtatttttattttccatgtttttag
taacttccatatcaatttagcactagaagataactttttttaggact
cggtaaaccatacaagtagggtcatactttatcgtttatccattaat
gtatatccataaattcactgattatgcggtatttcctttgttacact
gtcttgaacaagtattagtacatgtagtttcttaaagattgtttaat
caaccaaaaagattgaaactttgcag
HaHB1 protein
SEQ ID NO: 5
MTCTGMAFFSSNFMLQSSQEDDHHAPTSLSPILPPCSTTTQDFSGAA
FLGKRSMSSYSGLNNNNMDGCDQEGNMNGEDELSDDGSQLLAGEKKR
RLNMEQVKTLERNFELGNKLEPERKMQLARALGLQPRQIAIWFQNRR
ARWKTKQLEKDYDALKRQFEAVKAENDSLQSQNHKLHAEIMALKNRE
PAELINLNIKETEGSCSNRSENSSEIKLDISRTPATDSPLSSHHQHQ
HQPIPNLFPSSNIDRPNSNNIVAHQLFHNSSSRPADHQLHCHKLDQS
NAIKEECFSTMFVGMDDQSGFWPWLEQPQFN
HaHB1 cDNA
SEQ ID NO: 6
ttgaaattctgagaaaagccacataatcaaagctaaagaggtggttt
aaacagctgATGACTTGCACTGGAATGGCTTTCTTCTCCTCCAATTT
CATGTTACAATCCTCCCAAGAAGATGACCATCATGCCCCTACATCTC
TCTCTCCAATCCTCCCACCTTGCAGTACCACCACTCAAGATTTCAGT
GGTGCTGCTTTCTTGGGAAAAAGATCTATGTCTTCTTACTCAGGTTT
GAACAACAACAACATGGATGGATGTGATCAAGAAGGGAACATGAATG
GAGAAGATGAGTTATCAGATGATGGATCACAGCTTCTTGCAGGAGAG
AAAAAGAGGAGATTAAACATGGAACAAGTGAAGACACTTGAGAGAAA
CTTTGAGTTAGGAAATAAGCTTGAACCTGAGAGGAAAATGCAACTTG
CAAGAGCACTTGGACTACAACCAAGACAGATTGCTATATGGTTTCAA
AACAGAAGAGCTAGATGGAAAACTAAACAGTTGGAAAAAGACTATGA
TGCCCTCAAGAGACAGTTTGAAGCTGTTAAAGCTGAGAATGATTCAC
TCCAATCTCAAAATCATAAACTTCATGCTGAGATAATGGCACTAAAA
AATAGGGAGCCAGCAGAACTAATCAACCTCAACATAAAAGAAACAGA
AGGATCTTGCAGCAACCGAAGCGAAAACAGCTCTGAAATCAAACTAG
ACATCTCAAGAACACCGGCTACCGATAGCCCTTTATCATCACACCAT
CAACACCAACACCAGCCAATACCTAATCTTTTTCCATCGTCGAATAT
CGATAGGCCTAATTCGAATAACATTGTGGCGCATCAACTTTTCCACA
ATTCGTCATCAAGGCCGGCAGATCATCAACTTCATTGCCACAAACTC
GATCAATCGAATGCCATTAAAGAAGAATGTTTTAGCACAATGTTTGT
TGGTATGGATGATCAATCAGGGTTTTGGCCATGGTTGGAACAACCAC
AATTCAATTGAtggaatcaagaagcaaaaaagcaaaagaaaacggta
cccgattcgccttcttggctttggtttgattatattaaagatggaga
tcatcaatctgtttgttctctaagctttaaattcttgttttttggta
cttaaattaatagagtaaaaattagaagaaaaaacgtattattattt
taaattcaagattagtgttt

Coding sequence: in upper case
5′ and 3′ untranslated regions: in lower case
Flanking nucleotides from first and second introns: in bold and underlined

HaHB1 gene sequence
SEQ ID NO: 7
gtcgagctcgtctcgtaaaatgttcgagtcagctccaattaaatcat
gtcggcttgttatattttttttaatttatttttgatattttttacat
atatttataacataaaaaacaaaataaaaataaaaattacacatata
tctatatgtattatttttctaaaattttaaatagcgaaagacatatt
aaaagtattatatgtataattttgtttagcttcccatatttttatat
gttattaattaattaaacttaaaatgttaacactttaacacctctta
catactttttagttcaatacatttaaaattaaaattaatctatatgc
aataaaataattcaagcaggcttgcaagctcacgagtcgagccatgc
ctggctcgagctcgactcatttacaaatcgagccaccaagccgactc
gtttataaccgagttttttttagccgagtttttttcaagcgaacttc
aaacaagtcacgcgcttttaattaacacattctagtctaaagaagat
aattgaaagagaaagtagatataagtaaaaggagtagccaaagatat
aaatttagggtctaaacaacctaatattgtttaattttttttaaata
aactagtttttttttaccgattatctgtgttatatgtcttagtttga
catgataagttatcataattacttgtagtatttttatatcagaaata
tacgttggagaattaaattttatcctgatcgtcaattgacaagaaca
aaaatcaacatctcatggttttttactaatttatatgattaaagata
tatggttgtaagaaaaagaacaatgtacatcaaatggtgaaatttga
atatttgatagtaacgtaatccattgtgtatttcttattattttatc
attttcccaaggtgtgtcatatatagtgtctccattctttctatagc
acaatatccttcacctccctctctctctctctctctctaaaaatgat
gatgagacgacaaagatcgaattcttgaaattctgagaaaagccaca
taatcaaagctaaagaggtggtttaaacagctgATGACTTGCACTGG
AATGGCTTTCTTCTCCTCCAATTTCATGTTACAATCCTCCCAAGAAG
ATGACCATCATGCCCCTACATCTCTCTCTCCAATCCTCCCACCTTGC
AGTACCACCACTCAAGATTTCAGTGGTaattaactcaccttaactaa
gttacttatgacaacatctctctcatagatcttgatgcagcttgcat
tcatgagttgtgatgtacaactcattcatgcattagggtttcagttt
tttcaaagttttttttttattttttcttctgtttcaagatcatgatg
atgagttgtgctgaacacttgaacagctcattgatgcattagggttt
gttttagtttcaagttctttcttttctttcattttcatgcactaaat
ccatatgggcttgaagaaagtttgaatctttatatgttagttgatga
tcttgatgcaggtGCTGCTTTCTTGGGAAAAAGATCTATGTCTTCTT
ACTCAGGTTTGAACAACAACAACATGGATGGATGTGATCAAGAAGGG
AACATGAATGGAGAAGATGAGTTATCAGATGATGGATCACAGCTTCT
TGCAGGAGAGAAAAAGAGGAGATTAAACATGGAACAAGTGAAGACAC
TTGAGAGAAACTTTGAGTTAGGAAATAAGCTTGAACCTGAGAGGAAA
ATGCAACTTGCAAGAGCACTTGGACTACAACCAAGACAGATTGCTAT
ATGGTTTCAAAACAGAAGAGCTAGATGGAAAACTAAACAGTTGGAAA
AAGACTATGATGCCCTCAAGAGACAGTTTGAAGCTGTTAAAGCTGAG
AATGATTCACTCCAATCTCAAAATCATAAACTTCATGCTGgtaatat
tagtttgattgtttattgcatctatcaatcattagattctactcttt
acttgatcacacagaaagtaactaaaccttttttcctaatgataaca
atatttgttttgcaaatctaatggcaatcaaataaaagtttctggta
agcagccatgatctatttatttttcactatttgagtaagtttaaaag
ttgcatttatcctcactaattatatacaacactaaaataatcattaa
actgactgttataattactttccgtaaacggtatgccaaaacttaaa
atgattaacaattttataagaatggaaagtaaaatcattacactatt
tcccatattagtcatgaccaaagtttgtttctttctgaagggcaaaa
gggtcaatatgcttatatgcagcatgggcaaaagaagtagagtgtat
atcaaaattcatatctttattttcttttcaaagtttaggtaacaaaa
agaagaaattataaacgagtttgttacaattccacaagtacatgaag
aaacaaaatttgttagtatttttattttccatgtttttagtaacttc
catatcaatttagcactagaagataactttttttaggactcggtaaa
ccatacaagtagggtcatactttatcgtttatccattaatgtatatc
cataaattcactgattatgcggtatttcctttgttacactgtcttga
acaagtattagtacatgtagtttcttaaagattgtttaatcaaccaa
aaagattgaaactttgcagAGATAATGGCACTAAAAAATAGGGAGCC
AGCAGAACTAATCAACCTCAACATAAAAGAAACAGAAGGATCTTGCA
GCAACCGAAGCGAAAACAGCTCTGAAATCAAACTAGACATCTCAAGA
ACACCGGCTACCGATAGCCCTTTATCATCACACCATCAACACCAACA
CCAGCCAATACCTAATCTTTTTCCATCGTCGAATATCGATAGGCCTA
ATTCGAATAACATTGTGGCGCATCAACTTTTCCACAATTCGTCATCA
AGGCCGGCAGATCATCAACTTCATTGCCACAAACTCGATCAATCGAA
TGCCATTAAAGAAGAATGTTTTAGCACAATGTTTGTTGGTATGGATG
ATCAATCAGGGTTTTGGCCATGGTTGGAACAACCACAATTCAATTGA
tggaatcaagaagcaaaaaagcaaaagaaaacggtacccgattcgcc
ttcttggctttggtttgattatattaaagatggagatcatcaatctg
tttgttctctaagctttaaattcttgttttttggtacttaaattaat
agagtaaaaattagaagaaaaaacgtattattattttaaattcaaga
ttagtgttt

Coding sequence: in upper case
Promoter region: lower case
5′ and 3′ untranslated regions: in underlined lower case
First and second introns: in bold lower case

C-terminus of HaHB1
SEQ ID NO: 8
LINLNIKETEGSCSNRSENSSEIKLDISRTPATDSPLSSHHQHQHQP
IPNLFPSSNIDRPNSNNIVAHQLFHNSSSRPADHQLHCHKLDQSNAI
KEECFSTMFVGMDDQSGFWPWLEQPQFN
HAaHB1 C-terminus motif I (CI)
SEQ ID NO: 9
LINLNIKETEGSCSNRSENSSEIKLDISRTPATDS
HaHB1 C-terminus motif II (CII)
SEQ ID NO: 10
IKEECFSTMFVGMDDQSGFWPWLEQPQFN
Consensus sequence for the conserved region
adjacent to the leucine zipper
(CI, 34 aminoacids)
SEQ ID NO: 11
SINLNKETEGSCSNRSENSSDIKLDISRTPAIDS
Consensus sequence for the second conserved
region located at the C-terminal end
(CII, 29 aminoacids)
SEQ ID NO: 12
VKEESLSNMFCGIDDQSGFWPWLEQQHFN
N-terminus of HaHB1
SEQ ID NO: 13
MTCTGMAFFSSNFMLQSSQEDDHHAPTSLSPILPPCSTTTQDFSGAA
FLGKRSMSSYSGLNNNNMDGCDQEGNMNGEDELSDDGSQLLAGE
N-terminal homeodomain consensus sequence
SEQ ID NO: 14
KKRRLTDEQVKALEKSFELENKLEPERKVQLARELGLQPRQVAVWFQ
NRRARWKTKQ
ATHB13
SEQ ID NO: 15:
ACCAGAAGTGGTATAGTCTAGGCCGATACATTTCACTATCTCTCTCT
CTTTTGTTTTTCCTCTTCTTCTTTTTTTCCATTTGATTTCAAACTCT
CACACAAAGAGCTTCAGATTTATAAGACCATGATAATGGCTTTAAGA
CAAAGATTGGCAAGAAGAAAAAACTAAAGAGAAACGACCAAAATCTC
AAGCAAACAGTACTAACTTCTGTTGCAAAACAGAAGAAGATGTCTTG
TAATAATGGAATGTCTTTTTTCCCTTCAAATTTCATGATCCAAACCT
CTTACGAAGATGATCATCCTCATCAATCTCCATCTCTTGCTCCTCTT
CTTCCTTCTTGCTCTCTACCTCAAGATCTCCATGGTATATATACATA
AACTTCCACACACATCTCCTCTGTTTTCTCTCTATCTCTTTCTAATG
CTCTGTTCTGTTCTGTTTCAGGATTTGCTTCGTTTCTAGGTAAGAGA
TCTCCAATGGAAGGGTGTTGTGATTTAGAAACAGGGAACAATATGAA
TGGAGAAGAGGATTATTCAGATGATGGGTCACAAATGGGAGAGAAGA
AGAGGAGATTGAACATGGAACAAGTGAAGACACTAGAGAAGAACTTT
GAGCTTGGAAACAAACTTGAACCAGAGAGGAAAATGCAGCTAGCTCG
TGCCTTAGGTTTGCAACCAAGACAGATCGCGATTTGGTTTCAAAATC
GAAGAGCTCGTTGGAAAACAAAGCAGCTAGAGAAAGATTATGATACT
CTTAAACGACAGTTTGATACACTTAAAGCTGAAAATGATCTTCTTCA
AACTCATAATCAGAAACTCCAAGCTGAGGTAATTAATCTCATAAATT
AACAAAAAAAATCAATATGTGTTATTTTTTTTTGGGTTAATGATCAA
TAATTACAGTTATTTTCCATCTAAAGGATGATTTTTTTCTTTTTAAA
AAAGGTTAAAAATTATATTTCTGGTTTATAATTATTTGGATCAGGAG
TTGCTTTCAGGTAGGGTTAAAAAACTGGACATGATTCATGACTTTTC
AGACATCATTATCTCTTTTTTTCTTCACTCTTGTCTGGAAAGAGATC
TGAAAACAATAGTTTCTTTATGCTTATCACATTGTACAGTAACTCTG
TTTATGTTTAAAATTTTGTCTTTAATTACGCAGATAATGGGATTAAA
AAACAGAGAACAAACAGAATCAATAAATCTAAACAAAGAAACTGAAG
GATCTTGCAGTAACAGAAGTGATAACAGTTCAGATAATCTCAGACTA
GATATCTCAACTGCGCCGCCATCAAACGACAGTACATTAACCGGTGG
CCACCCACCGCCACCACAGACAGTTGGTCGACACTTCTTCCCACCGT
CGCCAGCCACCGCAACGACAACTACTACAACAATGCAGTTCTTTCAA
AACTCATCTTCAGGACAGAGTATGGTTAAAGAAGAGAATAGTATCAG
TAACATGTTCTGTGCAATGGATGACCATTCTGGTTTTTGGCCATGGC
TTGATCAGCAACAGTACAATTGAAATTGGTCTACCTGTTTTTTTTGT
TTTTGTTTTTAAAAAAATTTATATTTTTTTTTTTTGTATTTGGAATT
TTGATCAGAAGAACCCATGCATGTTTTCAAAAACTGGAATCTATATC
ATTAGCTCACTTTGAAATCTGCAACCAAACACCACTGAGGTTTTTTG
TTTACTTTTTGAGTAAATGAGATGTAAAAAAATGGGTAATATCCATT
ATATTATATAAAAAATAATATCATTATGGCCCAACATTTTTCTGTAT
GGAGAAAAATAAAATAAATTGTATATT
ATHB13 protein
SEQ ID NO: 16:
mscnngmsffpsnfmiqtsyeddhphqspslapllpscslpqdlhgf
asflgkrspmegccdletgnnmngeedysddgsqmgekkrrlnmeqv
ktleknfelgnkleperkmqlaralglqprqiaiwfqnrrarwktkq
lekdydtlkrqfdtlkaendllqthnqklqaeimglknreqtesinl
nketegscsnrsdnssdnlrldistappsndstltgghppppqtygr
hffppspatattttttmqffqnsssggsmykeensisnmfcamddhs
gfwpwldqqqyn

1. A method for increasing yield of a plant under moderate stress conditions, the method comprising introducing and expressing in the plant a nucleic acid sequence comprising SEQ ID NO:2, 6 or 7, a functional part of SEQ ID NO:2, 6, or 7, or a homologue or functional variant of SEQ ID NO:2, 6, or 7.

2. The method of claim 2, wherein the functional part is a nucleic acid sequence encoding the polypeptide of SEQ ID NO:8.

3. A method of claim 1, further comprising transforming the plant with a nucleic acid sequence encoding AtHB13 comprising SEQ ID NO:15.

4. The method of claim 1, wherein the functional variant is a chimeric nucleic acid construct comprising a nucleic acid sequence encoding the N-terminal sequence of an HD Zip protein comprising the HD-Zip domain of subfamily I or a sequence comprising an N-terminal consensus motif or part thereof operatively associated with a nucleic acid sequence encoding a sequence comprising the C-terminus of HaHB1, or a sequence comprising a C-terminal consensus motif or part thereof.

5. The method of claim 4, wherein the C-terminal sequence comprises SEQ ID NO:8 or one or more of SEQ ID NOs:9, 10, 11 or 12.

6. The method of claim 4, wherein the C-terminal sequence comprises a sequence with at least 80%, at least 90%, or at least 95% homology to the consensus sequence of SEQ ID NO:8.

7. The method of claim 6, wherein the C-terminal sequence comprises a sequence with at least 95%, 96%, 97%, 98% or 99% homology to SEQ ID NO:8.

8. The method of claim 4, wherein the N-terminal sequence comprises a sequence with at least 80% homology to the consensus sequence of SEQ ID NO:14.

9. The method of claim 8, wherein the N-terminal sequence comprises a sequence with at least 90% homology, or at least 95% homology to SEQ ID NO:14.

10. The method of claim 9, wherein the homology is at least 95%, 96%, 97%, 98% or 99%.

11. The method of claim 1, wherein the moderate stress conditions include drought, freezing, low-temperature, chilling, high salinity and/or invading pathogens.

12. The method of claim 11, wherein the moderate stress conditions include drought.

13. The method of claim 11, wherein the moderate stress conditions include high salinity.

14. The method of claim 1, wherein the plant is a crop plant.

15. A method for producing a plant with increased yield under moderate stress conditions, the method comprising introducing and expressing in the plant a nucleic acid sequence comprising SEQ ID NO:2, 6 or 7, a functional part of SEQ ID NO:2, 6, or 7, or a homologue or functional variant of SEQ ID NO:2, 6, or 7.

16. The method of claim 1, wherein the nucleic acid comprises a regulatory sequence.

17. The method of claim 16, wherein the regulatory sequence comprises SEQ ID NO:1.

18. (canceled)
